SEQUENCE LISTING (62 sequences)

SEQ ID NO: 1 - DNA, 21 bp, Artificial Sequence
  Description of Artificial Sequence Synthetic primer
  modified_base (1)..(1): PO4-C
  modified_base (4)..(4): a, c, g, t, unknown or other
ctantggacagcaagcgaacc

SEQ ID NO: 2 - DNA, 26 bp, Artificial Sequence
  Description of Artificial Sequence Synthetic primer
  modified_base (1)..(1): PO4-A
  modified_base (4)..(4): a, c, g, t, unknown or other
aatntcagaagaactcgtcaagaagg

SEQ ID NO: 3 - DNA, 25 bp, Artificial Sequence
  Description of Artificial Sequence Synthetic primer
  modified_base (1)..(1): PO4-G
gctctagatggacagcaagcgaacc

SEQ ID NO: 4 - DNA, 29 bp, Artificial Sequence
  Description of Artificial Sequence Synthetic primer
  modified_base (1)..(1): PO4-G
ggaattctcagaagaactcgtcaagaagg

SEQ ID NO: 5 - DNA, 29 bp, Artificial Sequence
  Description of Artificial Sequence Synthetic primer
  modified_base (1)..(1): PO4-G
  modified_base (12)..(12): a, c, g, t, unknown or other
ggtattgagggntggacagcaagcgaacc

SEQ ID NO: 6 - DNA, 37 bp, Artificial Sequence
  Description of Artificial Sequence Synthetic primer
  modified_base (1)..(1): PO4-A
  modified_base (15)..(15): a, c, g, t, unknown or other
agaggagagttagantcagaagaactcgtcaagaagg

SEQ ID NO: 7 - DNA, 20 bp, Artificial Sequence
  Description of Artificial Sequence Synthetic primer
aaatgcggttaacctcgcag

SEQ ID NO: 8 - DNA, 20 bp, Artificial Sequence
  Description of Artificial Sequence Synthetic primer
  Description of DNA/RNA Hybrid Sequence Synthetic primer
accutggaatcacctccccc

SEQ ID NO: 9 - DNA, 20 bp, Artificial Sequence
  Description of Artificial Sequence Synthetic primer
  Description of DNA/RNA Hybrid Sequence Synthetic primer
aggutggtatcaaggttaca

SEQ ID NO: 10 - DNA, 20 bp, Artificial Sequence
  Description of Artificial Sequence Synthetic primer
  Description of DNA/RNA Hybrid Sequence Synthetic primer
cuaagggtgggaaaatagac

SEQ ID NO: 11 - DNA, 20 bp, Artificial Sequence
  Description of Artificial Sequence Synthetic primer
agaaccccagtaaatcttgc

SEQ ID NO: 12 - DNA, 20 bp, Artificial Sequence
  Description of Artificial Sequence Synthetic primer
cttacttccagagtccttgg

SEQ ID NO: 13 - DNA, 20 bp, Artificial Sequence
  Description of Artificial Sequence Synthetic primer
  Description of DNA/RNA Hybrid Sequence Synthetic primer
uucuaagggtgggaaaatag

SEQ ID NO: 14 - DNA, 20 bp, Artificial Sequence
  Description of Artificial Sequence Synthetic primer
agaaccccagtaaatcttgc

SEQ ID NO: 15 - DNA, 22 bp, Artificial Sequence
  Description of Artificial Sequence Synthetic oligonucleotide
  modified_base (1)..(1): PO4-T
  modified_base (11)..(11): a, c, g, t, unknown or other
tcgacatatgnctacctaccta

SEQ ID NO: 16 - DNA, 22 bp, Artificial Sequence
  Description of Artificial Sequence Synthetic oligonucleotide
  modified_base (1)..(1): PO4-G
gatctaggtaggtagtcatatg

SEQ ID NO: 17 - DNA, 22 bp, Artificial Sequence
  Description of Artificial Sequence Synthetic oligonucleotide
  modified_base (1)..(1): PO4-T
  modified_base (11)..(11): a, c, g, t, unknown or other
tcgacatatgnctacctaccta

SEQ ID NO: 18 - DNA, 22 bp, Artificial Sequence
  Description of Artificial Sequence Synthetic oligonucleotide
  modified_base (1)..(1): PO4-G
gatctaggtaggtagccatatg

SEQ ID NO: 19 - DNA, 22 bp, Artificial Sequence
  Description of Artificial Sequence Synthetic oligonucleotide
  modified_base (1)..(1): PO4-T
tcgacatatgactacctaccta

SEQ ID NO: 20 - DNA, 22 bp, Artificial Sequence
  Description of Artificial Sequence Synthetic oligonucleotide
  modified_base (1)..(1): PO4-G
  modified_base (16)..(16): a, c, g, t, unknown or other
gatctaggtaggtagncatatg

SEQ ID NO: 21 - DNA, 22 bp, Artificial Sequence
  Description of Artificial Sequence Synthetic oligonucleotide
  modified_base (1)..(1): PO4-T
tcgacatatggctacctaccta

SEQ ID NO: 22 - DNA, 22 bp, Artificial Sequence
  Description of Artificial Sequence Synthetic oligonucleotide
  modified_base (1)..(1): PO4-G
  modified_base (16)..(16): a, c, g, t, unknown or other
gatctaggtaggtagncatatg

SEQ ID NO: 23 - DNA, 22 bp, Artificial Sequence
  Description of Artificial Sequence Synthetic oligonucleotide
  modified_base (1)..(1): PO4-T
  modified_base (11)..(11): a, c, g, t, unknown or other
tcgacatatgnctacctaccta

SEQ ID NO: 24 - DNA, 22 bp, Artificial Sequence
  Description of Artificial Sequence Synthetic oligonucleotide
  modified_base (1)..(1): PO4-G
  modified_base (16)..(16): a, c, g, t, unknown or other
gatctaggtaggtagncatatg

SEQ ID NO: 25 - DNA, 22 bp, Artificial Sequence
  Description of Artificial Sequence Synthetic oligonucleotide
  modified_base (1)..(1): PO4-T
  modified_base (11)..(11): a, c, g, t, unknown or other
tcgacatatgnctacctaccta

SEQ ID NO: 26 - DNA, 22 bp, Artificial Sequence
  Description of Artificial Sequence Synthetic oligonucleotide
  modified_base (1)..(1): PO4-G
  modified_base (16)..(16): a, c, g, t, unknown or other
gatctaggtaggtagncatatg

SEQ ID NO: 27 - DNA, 22 bp, Artificial Sequence
  Description of Artificial Sequence Synthetic oligonucleotide
  modified_base (1)..(1): PO4-G
  modified_base (16)..(16): a, c, g, t, unknown or other
gatctaggtaggtagncatatg

SEQ ID NO: 28 - DNA, 22 bp, Artificial Sequence
  Description of Artificial Sequence Synthetic oligonucleotide
  modified_base (1)..(1): PO4-T
  modified_base (11)..(11): a, c, g, t, unknown or other
tcgacatatgnctacctaccta

SEQ ID NO: 29 - DNA, 22 bp, Artificial Sequence
  Description of Artificial Sequence Synthetic oligonucleotide
  modified_base (1)..(1): PO4-T
  modified_base (11)..(11): a, c, g, t, unknown or other
tcgacatatgnctacctaccta

SEQ ID NO: 30 - DNA, 22 bp, Artificial Sequence
  Description of Artificial Sequence Synthetic oligonucleotide
  modified_base (1)..(1): PO4-G
  modified_base (16)..(16): a, c, g, t, unknown or other
gatctaggtaggtagncatatg

SEQ ID NO: 31 - DNA, 22 bp, Artificial Sequence
  Description of Artificial Sequence Synthetic oligonucleotide
  modified_base (1)..(1): PO4-T
  modified_base (11)..(11): a, c, g, t, unknown or other
tcgacatatgnctacctaccta

SEQ ID NO: 32 - DNA, 22 bp, Artificial Sequence
  Description of Artificial Sequence Synthetic oligonucleotide
  modified_base (1)..(1): PO4-G
gatctaggtaggtagtcatatg

SEQ ID NO: 33 - DNA, 22 bp, Artificial Sequence
  Description of Artificial Sequence Synthetic oligonucleotide
  modified_base (1)..(1): PO4-T
  modified_base (11)..(11): a, c, g, t, unknown or other
tcgacatatgnctacctaccta

SEQ ID NO: 34 - DNA, 22 bp, Artificial Sequence
  Description of Artificial Sequence Synthetic oligonucleotide
  modified_base (1)..(1): PO4-G
gatctaggtaggtagccatatg

SEQ ID NO: 35 - DNA, 22 bp, Artificial Sequence
  Description of Artificial Sequence Synthetic oligonucleotide
  modified_base (1)..(1): PO4-T
tcgacatatgactacctaccta

SEQ ID NO: 36 - DNA, 22 bp, Artificial Sequence
  Description of Artificial Sequence Synthetic oligonucleotide
  modified_base (1)..(1): PO4-G
  modified_base (16)..(16): a, c, g, t, unknown or other
gatctaggtaggtagncatatg

SEQ ID NO: 37 - DNA, 22 bp, Artificial Sequence
  Description of Artificial Sequence Synthetic oligonucleotide
  modified_base (1)..(1): PO4-T
tcgacatatggctacctaccta

SEQ ID NO: 38 - DNA, 22 bp, Artificial Sequence
  Description of Artificial Sequence Synthetic oligonucleotide
  modified_base (1)..(1): PO4-G
  modified_base (16)..(16): a, c, g, t, unknown or other
gatctaggtaggtagncatatg

SEQ ID NO: 39 - DNA, 31 bp, Artificial Sequence
  Description of Artificial Sequence Synthetic oligonucleotide
  modified_base (1)..(1): PO4-G
  modified_base (14)..(14): a, c, g, t, unknown or other
gaggagaagcccgntcactccttggcggaga

SEQ ID NO: 40 - DNA, 35 bp, Artificial Sequence
  Description of Artificial Sequence Synthetic oligonucleotide
  modified_base (1)..(1): PO4-A
  modified_base (13)..(13): a, c, g, t, unknown or other
acttgtcgtcgtntaccgagctcgaattcgtaatc

SEQ ID NO: 41 - DNA, 34 bp, Artificial Sequence
  Description of Artificial Sequence Synthetic oligonucleotide
  modified_base (1)..(1): PO4-A
  modified_base (14)..(14): a, c, g, t, unknown or other
acgggcttctcctnatcctctagagtcgacctgc

SEQ ID NO: 42 - DNA, 36 bp, Artificial Sequence
  Description of Artificial Sequence Synthetic oligonucleotide
  modified_base (1)..(1): PO4-G
  modified_base (13)..(13): a, c, g, t, unknown or other
gacgacgacaagntgttgacaattaatcatcggctc

SEQ ID NO: 43 - DNA, 36 bp, Artificial Sequence
  Description of Artificial Sequence Synthetic oligonucleotide
  modified_base (1)..(1): PO4-G
  modified_base (14)..(14): a, c, g, t, unknown or other
gaggagaagcccgntcagaagaactcgtcaagaagg

SEQ ID NO: 44 - DNA, 35 bp, Artificial Sequence; identical to SEQ ID NO: 40
  Description of Artificial Sequence Synthetic oligonucleotide
  modified_base (1)..(1): PO4-A
  modified_base (13)..(13): a, c, g, t, unknown or other

SEQ ID NO: 45 - DNA, 34 bp, Artificial Sequence; identical to SEQ ID NO: 41
  Description of Artificial Sequence Synthetic oligonucleotide
  modified_base (1)..(1): PO4-A
  modified_base (14)..(14): a, c, g, t, unknown or other

SEQ ID NO: 46 - DNA, 30 bp, Artificial Sequence
  Description of Artificial Sequence Synthetic oligonucleotide
  modified_base (1)..(1): PO4-G
  modified_base (13)..(13): a, c, g, t, unknown or other
gacgacgacaagntggacagcaagcgaacc

SEQ ID NO: 47 - DNA, 21 bp, Artificial Sequence
  Description of Artificial Sequence Synthetic oligonucleotide
  modified_base (1)..(1): PO4-C
  modified_base (4)..(4): a, c, g, t, unknown or other
ctantggacagcaagcgaacc

SEQ ID NO: 48 - DNA, 21 bp, Artificial Sequence
  Description of Artificial Sequence Synthetic oligonucleotide
tggacagcaagcgaaccggaa

SEQ ID NO: 49 - DNA, 26 bp, Artificial Sequence
  Description of Artificial Sequence Synthetic oligonucleotide
atcgccttcttgacgagttcttctga

SEQ ID NO: 50 - DNA, 26 bp, Artificial Sequence
  Description of Artificial Sequence Synthetic oligonucleotide
  modified_base (1)..(1): PO4-A
  modified_base (4)..(4): a, c, g, t, unknown or other
aatntcagaagaactcgtcaagaagg

SEQ ID NO: 51 - DNA, 26 bp, Artificial Sequence
  Description of Artificial Sequence Synthetic oligonucleotide
  modified_base (4)..(4): a, c, g, t, unknown or other
ctantggacagcaagagttcttctga

SEQ ID NO: 52 - DNA, 26 bp, Artificial Sequence
  Description of Artificial Sequence Synthetic oligonucleotide
  modified_base (1)..(1): PO4-A
  modified_base (4)..(4): a, c, g, t, unknown or other
aatntcagaagaactcttgctgtcca

SEQ ID NO: 53 - DNA, 29 bp, Artificial Sequence
  Description of Artificial Sequence Synthetic oligonucleotide
  modified_base (1)..(1): PO4-G
  modified_base (12)..(12): a, c, g, t, unknown or other
ggtattgagggntggacagcaagcgaacc

SEQ ID NO: 54 - DNA, 21 bp, Artificial Sequence
  Description of Artificial Sequence Synthetic oligonucleotide
tggacagcaagcgaaccggaa

SEQ ID NO: 55 - DNA, 26 bp, Artificial Sequence
  Description of Artificial Sequence Synthetic oligonucleotide
atcgccttcttgacgagttcttctga

SEQ ID NO: 56 - DNA, 37 bp, Artificial Sequence; identical to SEQ ID NO: 6
  Description of Artificial Sequence Synthetic oligonucleotide
  modified_base (1)..(1): PO4-A
  modified_base (15)..(15): a, c, g, t, unknown or other

SEQ ID NO: 57 - DNA, 36 bp, Artificial Sequence
  Description of Artificial Sequence Synthetic oligonucleotide
  modified_base (1)..(1): PO4-G
  modified_base (14)..(14): a, c, g, t, unknown or other
gacgacgacaagantggacagcaagagttcttctga

SEQ ID NO: 58 - DNA, 38 bp, Artificial Sequence
  Description of Artificial Sequence Synthetic oligonucleotide
  modified_base (1)..(1): PO4-T
  modified_base (16)..(16): a, c, g, t, unknown or other
tgaggagaagcccggntcagaagaactcttgctgtcca

SEQ ID NO: 59 - DNA, 16 bp, Artificial Sequence
  Description of Artificial Sequence Synthetic oligonucleotide
tcttgtcgtcgtcatc

SEQ ID NO: 60 - DNA, 15 bp, Artificial Sequence
  Description of Artificial Sequence Synthetic oligonucleotide
ccgggcttctcctca

SEQ ID NO: 61 - DNA, 18 bp, Artificial Sequence
  Description of Artificial Sequence Synthetic oligonucleotide
gatgacgacgacaagatg

SEQ ID NO: 62 - DNA, 18 bp, Artificial Sequence
  Description of Artificial Sequence Synthetic oligonucleotide
taaccgggcttctcctca

(19) United States
(12) Patent Application Publication
(10) Pub. No.: US 2007/0264694 A1
(43) Pub. Date: Nov. 15, 2007

(54) USE OF NON-STANDARD BASES AND PROXIMITY EFFECTS FOR GENE ASSEMBLY AND CONVERSION OF NON-STANDARD BASES TO STANDARD BASES DURING DNA SYNTHESIS

(75) Inventor: James R. Prudent, Madison, WI (US)

Correspondence Address: FOLEY & LARDNER LLP, 150 EAST GILMAN STREET, P.O. BOX 1497, MADISON, WI, 53701-1497, US

(73) Assignee: EraGen Biosciences, Inc.

(21) Appl. No.: 11/733,011

(22) Filed: Apr. 9, 2007

Related U.S. Application Data
(60) Provisional application No. 60/790,460, filed on Apr. 7, 2006.

(51) Int. Cl. C12P 19/34

(52) U.S. Cl. 435/91.2

(57) ABSTRACT

The present methods relate to generating nucleic acid molecules using non-natural nucleotides. In some methods, the nucleic acid molecules may be generated by hybridizing a plurality of oligonucleotides comprising one or more non-natural nucleotides and using a polymerase and/or a coupling agent to link the hybridized oligonucleotides. The methods also relate to the use of proximity effects to generate nucleic acid molecules using non-natural nucleotides. Furthermore, the methods relate to the use of at least one non-natural base in a DNA template in order to generate a replicate of the DNA template in which the non-natural base has been replaced with a natural base.

### CROSS-REFERENCE TO RELATED PATENT APPLICATIONS

This application claims the benefit of U.S. Provisional Application No. 60/790,460, filed Apr. 7, 2006, which is incorporated by reference in its entirety.

### FIELD OF THE INVENTION

The methods disclosed herein pertain generally to the field of biology and particularly to techniques and methods for the synthesis, assembly and analysis of nucleic acid sequences using non-natural bases.

### BACKGROUND

Full-length genes or long, single or double-stranded nucleic acids of defined sequence (e.g., greater than 400 nucleotides) are important molecular tools used in diverse areas of biological and biochemical study. As such, gene synthesis methods have been developed as tools for a variety of studies including investigations related to gene function, structure-function relationship of proteins, codon optimization, construction of DNA vaccines, creation of synthetically engineered genomes and de novo synthesis of novel biopolymers, among others.

Some of the earliest methods of gene synthesis involved ligation reactions using small oligonucleotides (see e.g., Ecker, et al, J. Biol. Chem. 262(8):3524-3527, (1987)), or the use of restriction endonucleases such as FokI to sequentially cut and ligate various gene fragments into a vector to construct a whole gene (see, e.g., Mandecki and Bolling, Gene 68(1):101-107, (1988)). Methods using the polymerase chain reaction (“PCR”) were also developed, and variations on the theme of assembling single-stranded, overlapping oligonucleotides were explored and optimized (see, e.g., Ciccarelli et al., Nucleic Acids Res. 19(21):6007-6013, (1991); Strizhov et al., PNAS USA, 93:15012-15017, (1996); Young and Dong, Nucleic Acids Res. 32(7):e59, (2004); Smith et al., PNAS, 100(26):15440-15445, (2003); Rydzanicz, et al., Nucleic Acids Res. 33:W521-W525, (2005); Xiong, et al., Nucleic Acids Res., 32(12):e98, (2004)). However, key limitations common to these methods included cost, inefficiencies, sequence accuracy and speed. Tian, et al., Nature 432:1050-1053, (2004). Accordingly, efforts to improve these parameters have been undertaken, with a focus on high-fidelity polymerases, and on fast, cheap and accurate methods to synthesize short, precursor oligonucleotides.

The advent of nucleic acid chip technology and the ability to perform large scale parallel oligonucleotide synthesis reactions have provided a path for exploration. Development of methods incorporating the use of photolabile 5′ protecting groups, (e.g., Affymetrix or NimbleGen Systems, Inc. technology), ink-jet printing (e.g., Agilent Technologies methods), electronic acid/base arrays (such as used by Oxamer or Combimatrix Corp.), and photo-generated acid deprotection (such as Xeotron/Invitrogen methods) allows the synthesis of thousands of oligonucleotides on a single chip in parallel. Tian, et al., Nature 432:1050-1053, (2004).

However, even with these advances, a basic problem of complex gene assembly which involves annealing many short oligonucleotides sequences is the inability to obtain the level of hybridization specificity needed for accurate multi-oligonucleotide assembly. Additionally, factors such as the increase in error rate brought on by synthesizing large fragments, and the increase in error rate brought on by repetitive replication cycles required when shorter oligonucleotides are used, all contribute to higher costs and longer preparation times.

Accordingly, there is a need in the art for new methods of gene assembly and DNA synthesis.

### SUMMARY OF THE INVENTION

The present methods relate to the synthesis and assembly of single or double-stranded nucleic acid molecules using oligonucleotides comprising at least one non-natural base. In some methods, regions of the oligonucleotides may be complementary and anneal under annealing conditions; complementary regions may be at the 5′ and 3′ ends of the oligonucleotides. In some methods, the oligonucleotides may contain at least one non-natural base. In other methods, the at least one non-natural base may be in the complementary region of the oligonucleotides; in some methods, the at least one non-natural base of one oligonucleotide may base pair with another non-natural nucleotide in a complementary region of different oligonucleotide.

In some methods, one or more of a polymerase, a ligase or a polymerase chain reaction may be used to create a single or double-stranded molecule. In some methods involving a polymerase or a polymerase chain reaction, only natural mononucleotides may be provided for the reaction; in other methods, natural and non-natural mononucleotides may be provided.

For example, some methods may involve a first oligonucleotide comprising at least one non-natural nucleotide and a second oligonucleotide comprising at least one non-natural nucleotide. In some methods, the non-natural nucleotide or nucleotides in the first oligonucleotide may base-pair with the non-natural nucleotide or nucleotides in the second oligonucleotide. In some methods, at least one non-natural nucleotide is the terminal 5′ or the terminal 3′ nucleotide; in other methods, at least one non-natural nucleotide is within 5 nucleotides of the 5′ or 3′ terminal nucleotide.

In some methods, a first complement is synthesized. The first complement may be a complement to the first oligonucleotide that incorporates the second oligonucleotide, or the first complement may be a complement to the second oligonucleotide that incorporates the first oligonucleotide.

In other methods, the first complement can be hybridized to a third oligonucleotide that is different from the first oligonucleotide and the second oligonucleotide. The third oligonucleotide may include at least one non-natural nucleotide that is complementary to at least one non-natural nucleotide in the first complement. In some methods, the first and second oligonucleotide may be hybridized concurrently with the third oligonucleotide and the first complement.

A second complement may be synthesized in some methods. The second complement may be a complement to the third oligonucleotide that incorporates the first complement, or the second complement may be a complement to the first complement that incorporates the third oligonucleotide. In some methods, the first complement and the second complement may be synthesized concurrently.

In some methods, the hybridization of the first and second oligonucleotides, the synthesis of the first complement, the hybridization of the third oligonucleotide and the first complement, and the synthesis of the second complement may be performed sequentially.

In some methods, the complements may be covalently coupled. In other methods, the covalently linked complements may be amplified. For example, in some methods, covalently coupled oligonucleotides may be amplified using the polymerase chain reaction, or covalently coupled oligonucleotides may be replicated using a polymerase. Reaction mixture for amplification or replication may optionally include non-natural nucleotides.

The present methods also relate to the synthesis and assembly of single or double-stranded nucleic acid molecules using DNA oligomers that include at least one non-natural base and that may be ligated to oligonucleotides. For example, DNA oligomers that include at least one non-natural nucleotide may be ligated to a plurality of oligonucleotides to form tagged oligonucleotides. In some methods, a tagged oligonucleotide may hybridize to another tagged oligonucleotide. In some methods, the ligated oligomer sequences of the tagged oligonucleotides may contain at least one non-natural nucleotide that base-pairs with at least one non-natural nucleotide in another tagged oligomer.

In some methods, a first complement of at least one of the tagged oligonucleotides may be synthesized. For example, in some methods at least one of the tagged oligonucleotides may be used as a primer and may thereby be incorporated into the first complement of another tagged oligonucleotide. In some methods, a second complement may be synthesized. The second complement may be complementary to the first complement, and at least one of the tagged oligonucleotides may be used as a primer and thereby may be incorporated into the second complement. The following steps may be performed concurrently and/or repetitively. A synthesis reaction may optionally include one or more non-natural nucleotides.

In some methods, the complements (e.g., a first complement and a second complement) may be coupled. For example, a first complement and a second complement may be covalently linked by chemical or enzymatic methods. In some methods, covalently linked complements may be amplified, optionally using a reaction mixture that includes at least one non-natural nucleotide.

In some methods, at least one of the oligonucleotides, e.g., a “first oligonucleotide,” may be reversibly or irreversibly immobilized to a solid substrate at a first position and a “second oligonucleotide” may be reversibly or irreversibly immobilized on the solid substrate at a second position. The first position and second position may be proximal (i.e., closer to each other than to any other position). In some methods, the first and second positions may be at a distance of no more than between about 20 microns (preferably no more than 10 microns, even more preferably no more than 5 microns).

The methods also relate to the synthesis and assembly of relatively long (e.g., at least about 500 nucleotides, 1000 nucleotides, or 5000 nucleotides), single or double-stranded nucleic acid sequences using proximity effects. For example, the methods may include the synthesis and assembly of single or double-stranded nucleic acid molecules using oligonucleotides comprising at least one non-natural nucleotide that have been reversibly or irreversibly immobilized on a solid substrate. In some methods, the oligonucleotides may be reversibly immobilized by virtue of placement on a solid support, such as on a chip or a microchip. By way of example, but not by way of limitation, solid support synthesis methods that may involve reversibly immobilized oligonucleotides include Maskless Array Synthesis (see e.g., Richmond, et al., Nucleic Acid Res. 32:17 5011-5018 (2004)) and photolabile 5′ protecting groups and photolithographic processes.

In some methods, the oligonucleotides may be positioned on the solid support such that each oligonucleotide “X” is proximal to at least one other oligonucleotide “Y” where a region of oligonucleotide “X” is complementary to a region of oligonucleotide “Y.” In some methods, oligonucleotides X and Y contain at least one non-natural nucleotide, wherein the non-natural nucleotide or nucleotides in X are complementary to the non-natural nucleotides in Y. By placing oligonucleotides having complementarity at proximal positions, specific hybridization is facilitated.

In some methods, the oligonucleotides, which may comprise a plurality of oligonucleotides, may be about 5-25 nucleotides long. In other methods, the oligonucleotides may be about 25-100 nucleotides long, or the oligonucleotides may be about 100-200 nucleotides long. Typically, the oligonucleotides include a least one region that is complementary to a region on another oligonucleotide. An oligonucleotide may include two or more regions that are complementary to regions on one or more other oligonucleotides. The regions of complementarity may be about 1-10 nucleotides long, or in some instances about 10-25 nucleotides long. The region of complementarity on a first oligonucleotide may include one or more non-natural nucleotides, which optionally, may base pair with a non-natural nucleotide in another region of complementarity in a second oligonucleotide. In some methods, the 5′ region of a first oligonucleotide may be complementary to the 3′ region of a second oligonucleotide, and the 3′ region of a first oligonucleotide may be complementary to a 5′ region of a third oligonucleotide. In further methods, about one-half of a first oligonucleotide is complementary to about one-half of a second oligonucleotide, and the other about one-half of the first oligonucleotide is complementary to about one-half of a third oligonucleotide.

The at least one non-natural nucleotide may be present at any nucleotide position in an oligonucleotide. In some methods, the non-natural nucleotide is at about 1-3 nucleotides from the 5′ or the 3′ terminal end of the oligonucleotide or at 5′ or 3′ terminal end of the oligonucleotide, (or at about 4-10 nucleotides from the 5′ or the 3′ terminal end of the oligonucleotide, or about 10-25 nucleotides from the 5′ or the 3′ terminal end of the oligonucleotide).

In some methods, the non-natural nucleotide is selected from diCTP and diGTP. The non-natural nucleotide may include, for example isocytosine (iC), isoguanine (iG) or derivatives of these such as 5′-methylisocytosine. The non-natural nucleotide may include a self-pairing hydrophobic base such as described in McMinn et al., J. Am. Chem. Soc. 121:11585-11586, (1999), herein incorporated by reference. In other methods, the non-natural nucleotide may include 2-amino-6-(N,N-dimethylamino)purine and pyridine-2-one as described in Ohtsuki et al., PNAS 98(9):4922-4925, (2001). In still other methods, the non-natural nucleotides are benzo-homologated forms of adenine, guanine, cytosine, thymine and uracil as described by Gao et al., Angew. Chem. Int. Ed. 44:3118-3122, (2005).

In some methods the non-natural nucleotides comprise non-standard nucleobases that can pair with complementary non-standard nucleobases so as to fit the Watson-Crick geometry. For example, a resulting non-standard base pair may include a monocyclic six-membered ring pairing with a fused, bicyclic-heterocyclic ring system composed of a five-member ring fused with a six-member ring, with the orientation of the heterocycles with respect to each other and with respect to the backbone chain analogous to that found in DNA and RNA, but with the pattern of hydrogen bonds holding the base pair together different from that found the AT and GC base pairs. In some cases the non-standard bases may expand the size of the nucleic acid duplex, such as those described by Jianmin Gao, et al, Angew. Chem. Int. Ed. 2005, 44:3118-3122.

The present methods also relate to assembly of a full-length single or double-stranded nucleic acid sequence. For example, the methods may include coupling oligonucleotides that include at least one non-natural nucleotide in a ligase reaction. An oligonucleotide of the method typically has at least one region that is complementary to a corresponding region on a second oligonucleotide (e.g., a “template”). In some methods, an oligonucleotide may have at least two regions of complementarity, where the first region is complementary to a corresponding region on a second oligonucleotide and the second region is complementary to a corresponding region on a third oligonucleotide. In some methods, a region of complementarity may be fully complementary. In other methods, a region of complementarity may be partially complementary. Typically, regions of complementarity include at least about 90% complementarity (or at least about 95% complementarity). Typically, regions of complementarity will hybridize specifically under stringent conditions as known in the art.

In some methods, the complementary regions of a first oligonucleotide may include at least one non-natural nucleotide that is complementary to at least one non-natural nucleotide in the corresponding region of a second oligonucleotide. In some methods, complementary oligonucleotides may be hybridized to a contiguous portion of a template (e.g., “annealed to a template”) and coupled (e.g., enzymatically or chemically). In some methods, a ligase is used to covalently link oligonucleotides hybridized to a template. In some methods, the covalently linked oligonucleotides may be amplified using a reaction mixture that, optionally, includes non-natural nucleotides. Non-natural nucleotides may not be present in an amplification mixture.

In some methods, a plurality of oligonucleotides containing at least one non-natural base may be complementary to different regions of a single-stranded template that comprise a contiguous portion of the single-stranded template. In some methods, the oligonucleotides may be hybridized to the single-stranded template. In some methods, a ligase is used to covalently link the hybridized oligonucleotides to form a complement to the contiguous portion of the single-stranded template. The complement and/or single-stranded template may be amplified. In some methods, natural nucleotides may be used in the amplification reaction. In other methods, natural and non-natural mononucleotides may be used in the amplification reaction. The complement and/or single-stranded template may be transfected into a suitable cell for replications. For example, the complement and/or single-stranded template may be cloned into a suitable cloning vector, transfected into a cell, and replicated.

The present methods also relate to cloning, sequencing and expression of a single or double-stranded DNA assembled by the methods described herein. In some methods, the assembled sequence may be cloned into vector, which may include an expression vector. The vector may be transfected into a cell or an organism and the assembled sequence may be expressed in the cell or organism. In some methods, the assembled sequence may be recovered from the transfected cell and sequenced.

In some methods, the assembled single or double-stranded DNA is at least about 500 nucleotides (or at least about 1000 or 5000 nucleotides). In still other methods, the assembled single or double-stranded DNA sequence is at least about 10,000 bases.

The present methods also relates to conversion of specific non-natural nucleotides to natural nucleotides in a DNA template. For example, the methods may include synthesizing a DNA molecule with an A:T base pair at a selected position; the method may involve: synthesizing a DNA template that includes one of the following base pairs at a selected position: A:iC, iC:T, iG:iC, or iG:T and replicating the DNA template with a polymerase that converts iC:iG to T:A. Other methods relate to synthesizing a DNA molecule that may include a T:A base pair at a selected position, by replicating a DNA template that includes an iC:iG at the selected position, and replicating the DNA template with a polymerase that converts iC:iG to T:A. Other methods may include synthesizing a DNA molecule that may include a T:A or an A:T base pair at a selected position, by replicating a DNA template that includes an iC:iC, iG:iG or A:iG at the selected position, and replicating the DNA template with a polymerase that converts iC:iC, iG:iG or A:iG to T:A or A:T. Still other methods may include synthesizing a DNA molecule that may include a G:C or a T:A base pair at a selected position, by replicating a DNA template that includes an iC:C, G:iC or G:iG at the selected position, and replicating the DNA template with a polymerase that converts iC:C, G:iC or G:iG to G:C or T:A. Other methods may also include synthesizing a DNA molecule that may include a G:C or an A:T base pair at a selected position, by replicating a DNA template that includes an iG:C at the selected position, and replicating the DNA template with a polymerase that converts iG:C to G:C or A:T. In some methods, the DNA template may be replicated in a cell.

The present methods also relate to synthesizing a DNA molecule, including replicating a DNA template that includes at least one non-natural base in a cell. In some methods, the non-natural base may be iC and/or iG; in other methods the DNA template may include at least one base pair that includes at least one non-natural base, for example: iC:iG, iC:iC, iG:iG, iC:A, iC:C, iC:G, iC:T, iG:A, iG:C, iG:G, and iG:T. In some methods, the template is double stranded; in other methods, the DNA template may include 5′ or 3′ overhangs. In still other methods, the non-natural base may or may not be present in the 5′ or 3′ overhang. In still other methods, the DNA template may be present in a plasmid; for example, the template may be cloned into a plasmid. In some methods, a cell may be transfected with the plasmid. In some methods, the template may be amplified; in other methods the replicated molecule may be sequenced. In some methods the polynucleotide template may encodes at least a portion of a polypeptide.

Kits for performing any of the disclosed methods are also contemplated.

### BRIEF DESCRIPTION OF THE DRAWINGS

FIG. 1 shows the oligonucleotide combinations used to test the repair/conversion of DNA base-pair mismatches.

FIG. 2 illustrates the method of sequencing clones to detect the repair/conversion of DNA base-pair mismatches.

FIG. 3 shows the results of the repair/conversion of DNA base-pair mismatches.

FIG. 4 shows the results of the repair/conversion of DNA base-pair mismatches where mixed results are obtained.

FIG. 5 illustrates a scheme for ligation-independent cloning by generating overhangs having non-natural bases.

FIG. 6 illustrates a scheme for ligation-dependent cloning.

FIG. 7 shows the results of ligation dependent cloning using a variety of DNA polymerase enzymes.

FIG. 8 shows the directionality of inserts in a vector following ligation-dependent cloning.

FIG. 9 illustrates a scheme for ligation-independent cloning.

FIG. 10 shows the results of ligation-independent cloning following transformation of constructs with and without DNA ligase.

### DETAILED DESCRIPTION OF THE PREFERRED EMBODIMENTS

Disclosed herein are methods for the synthesis and assembly of single or double-stranded nucleic acid molecules. Some of the methods describe the use of oligonucleotides comprising non-natural bases to increase hybridization specificity in the assembly reactions.

Definitions

As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, the term “sample” is used in its broadest sense. A sample may include a bodily tissue or a bodily fluid including but not limited to blood (or a fraction of blood such as plasma or serum), lymph, mucus, tears, urine, and saliva. A sample may include an extract from a cell, a chromosome, organelle, or a virus. A sample may comprise DNA (e.g., genomic DNA), RNA (e.g., mRNA), and cDNA, any of which may be amplified to provide amplified nucleic acid. A sample may include nucleic acid in solution or bound to a substrate (e.g., as part of a microarray). A sample may comprise material obtained from an environmental locus (e.g., a body of water, soil, and the like) or material obtained from a fomite (i.e., an inanimate object that serves to transfer pathogens from one host to another).

The methods disclosed herein may include introducing a nucleotide template into a cell, which may include a eukaryotic cell and a prokaryotic cell. As used herein the terms “transformation” and “transfection” are meant to include the introduction of nucleic acid molecules into cells. The methods of the present invention may encompass both “transformation” and “transfection,” and it is understood that where one term is used in the specification in describing the methods, kits, procedures and compositions presented herein, the alternate term is also contemplated.

As used herein, the terms “converting,” “conversion” and “convert” mean that at least one nucleotide present in a reference polynucleotide (e.g., a template sequence) is changed in a replicated polynucleotide (e.g., using the reference polynucleotide as a template). “Converting” may include transitions (e.g., exchanging a purine for a purine or a pyrimidine for a pyrimidine) and transversions (e.g., exchanging a purine for a pyrimidine or a pyrimidine for a purine). “Converting” may also include the exchange of a non-natural nucleotide for a natural nucleotide, or the exchange of a natural nucleotide for a non-natural nucleotide.

As used herein, the term “microarray” refers to an arrangement of a plurality of polynucleotides, polypeptides, or other chemical compounds on a substrate. The terms “element” and “array element” refer to a polynucleotide, polypeptide, or other chemical compound having a unique and defined position on a microarray.

As used herein, an “oligonucleotide” is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides (“dNTP's”), which do not have a hydroxyl group at the 2′ position, and oligoribonucleotides (“NTP's”), which have a hydroxyl group in this position. Oligonucleotides also may include derivatives, in which the hydrogen of the hydroxyl group is replaced with organic groups, e.g., an allyl group. An “oligonucleotide” as used herein may contain natural and/or non-natural bases.

Oligonucleotides may be generated in any manner, including chemical synthesis, DNA replication, cloning, restriction of appropriate sequences, reverse transcription, PCR, or a combination thereof. For example, chemical synthesis methods can include the phosphotriester method described by Narang et al. Methods in Enzymology 68:90 (1979), the phosphodiester method disclosed by Brown et al. Methods in Enzymology 68:109 (1979), the diethylphosphoramidate method disclosed in Beaucage et al. Tetrahedron Letters 22:1859 (1981), and the solid support method disclosed in U.S. Pat. No. 4,458,066, all of which are incorporated herein by reference.

Oligonucleotides may also be synthesized on a chip, a microchip or other mass synthesis methods. By way of example but not by way of limitation, methods including the use of photolithographic methods as described by Richmond et al., Nucleic Acid Res. 32(17):5011-5018 (2004) and photo-generated acid deprotection methods as described by Gao, et al., Nucleic Acids Res. 29(22):4744-4750, (2001) may be used.

An oligonucleotide is a nucleic acid that includes at least two nucleotides. Oligonucleotides used in the methods disclosed herein typically include at least about twenty (20) nucleotides to about one-hundred (100) nucleotides. Preferred oligonucleotides for the methods disclosed herein include about 60-90 nucleotides. The exact size will depend on many factors, which in turn depend on the ultimate function or use of the oligonucleotide.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends.

An oligonucleotide may be designed to function as a “primer.” A “primer” as used herein is a short nucleic acid, usually a single stranded DNA oligonucleotide, which may be annealed to a target or template polynucleotide by complementary base-pairing. The primer may then be extended along the template DNA strand by a DNA polymerase enzyme. Primer pairs can be used for amplification of a nucleic acid sequence (e.g., by the polymerase chain reaction (PCR)).

An oligonucleotide may be designed to function as a “probe.” A “probe” refers to an oligonucleotide, its complements, or fragments thereof, which is used to detect identical, allelic or related nucleic acid sequences. Probes may include oligonucleotides which have been attached to a detectable label or reporter molecule. Typical labels include fluorescent dyes, radioactive isotopes, ligands, chemiluminescent agents, and enzymes.

In some embodiments, oligonucleotides as described herein may include a peptide backbone. For example, the oligonucleotides may include peptide nucleic acids or “PNA.” Peptide nucleic acids are described in WO 92/20702, which is incorporated herein by reference.

An oligonucleotide may be designed to be specific for a target or template nucleic acid sequence in a sample. For example, an oligonucleotide may be designed to include “antisense” nucleic acid sequence of the target or template nucleic acid. As used herein, the term “antisense” refers to any composition capable of base-pairing with the “sense” (coding) strand of a specific target nucleic acid sequence.

An antisense nucleic acid sequence may be “complementary” to a target or template nucleic acid sequence. As used herein, the terms “complementary” or “complementarity,” when used in reference to nucleic acids (i.e., a sequence of nucleotides such as an oligonucleotide or a target nucleic acid), refer to sequences that are related by base-pairing rules. For natural bases, the base pairing rules are those developed by Watson and Crick. For non-natural bases, as described herein, the base-pairing rules include the formation of hydrogen bonds in a manner similar to the Watson-Crick base pairing rules or by hydrophobic, entropic, steric or van der Waals forces.

The “complement of a nucleic acid sequence” as used herein refers to an oligonucleotide which, when aligned with the nucleic acid sequence such that the 5′ end of one sequence is paired with the 3′ end of the other, is in “antiparallel association.” As an example, for the sequence “5′-T-G-A-3′”, the complementary sequence is “3′-A-C-T-5′.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands has effects on the efficiency and strength of hybridization between the nucleic acid strands. Either term may also be used in reference to individual nucleotides (natural or non-natural), especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

Non-natural bases that are generally not considered “complementary” and are generally not considered “base-paired” include the non-natural bases that are non-specific or “universal.” Such universal bases can bind two or more generally naturally occurring bases in a relatively indiscriminate or non-preferential manner, with or without equal affinities. Examples of such non-specific or universal bases include 2′-deoxyinosine (inosine), 3′ nitropyrrole, 2′ deoxynucleoside (3′ nitropyrrole) and those disclosed in U.S. Pat. Nos. 5,438,131 and 5,681,947. Generally, when the base is “universal” for only a subset of the natural bases, that subset will generally be either purines (adenine or guanine) or pyrimidines (cytosine, thymine or uracil). Examples of nucleotides that can be considered universal for purines are known as the “K” base (N-6-methoxy-2,6-diaminopurine), as discussed in Bergstrom et al, Nucleic Acids Research 25: 1935 (1997), and pyrimidines are know as the “P” base (6H,8H-3,4-dihydropyrimido[4,5-c][1,2]oxazin-7-one), as discussed in Bergstrum et al., supra and U.S. Pat. No. 6,313,286. Other universal nucleotides include 5-nitroindole (5-nitroindole 2′ deoxynucleoside), 4-nitroindole (4-nitroindole 2′ deoxynucleoside), 6-nitroindole (6-nitroindole 2′-nucleoside or 2′-deoxynebularine).

As used herein a “complement” means a complementary copy of all or a part of an oligonucleotide sequence, including the primer used to prime the polymerase reaction. “Complement” also means a complementary copy of a nucleic acid molecule.

Oligonucleotides as described herein typically are capable of forming hydrogen bonds with oligonucleotides having a complementary base sequence. These bases may include the natural bases such as A, G, C, T and U, as well as artificial bases such as deaza-G. As described herein, a first sequence of an oligonucleotide is described as being 100% complementary with a second sequence of an oligonucleotide when the consecutive bases of the first sequence (read 5′->3′) follow the Watson-Crick rule of base pairing as compared to the consecutive bases of the second sequence (read 3′->5′). An oligonucleotide may include nucleotide substitutions. For example, a non-natural oligonucleotide may be used in place of a natural nucleotide such that the non-natural nucleotide exhibits a specific interaction that is similar to the natural base with another non-natural nucleotide.

An oligonucleotide that is specific for a target nucleic acid also may be specific for a nucleic acid sequence that has “homology” to the target nucleic acid sequence. As used herein, “homology” refers to sequence similarity or, interchangeably, sequence identity, between two or more polynucleotide sequences or two or more polypeptide sequences. The terms “percent identity” and “% identity” as applied to polynucleotide sequences, refer to the percentage of residue matches between at least two polynucleotide sequences aligned using a standardized algorithm (e.g., BLAST).

An oligonucleotide that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Equations for calculating Tₘ and conditions for nucleic acid hybridization are known in the art. Oligonucleotides used as specific primers for amplifying a target or template nucleic acid generally are capable of specifically hybridizing to the target nucleic acid.

As used herein, “nucleic acid,” “nucleotide sequence,” or “nucleic acid sequence” refer to a nucleotide, oligonucleotide, polynucleotide, or any fragment thereof and to naturally occurring or synthetic molecules. These phrases also refer to DNA or RNA of genomic or synthetic origin which may be single-stranded or double-stranded and may represent the sense or the antisense strand, or to any DNA-like or RNA-like material. An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. RNA may be used in the methods described herein and/or may be converted to cDNA by reverse-transcription for use in the methods described herein.

As used herein, “amplification” or “amplifying” refers to the production of additional copies of a nucleic acid sequence. Amplification is generally carried out using polymerase chain reaction (PCR) technologies known in the art. The term “amplification reaction system” refers to any in vitro means for multiplying the copies of a target sequence of nucleic acid. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, and nucleoside triphosphates, and optionally at least one labeled probe and/or optionally at least one agent for determining the melting temperature of an amplified target nucleic acid (e.g., a fluorescent intercalating agent that exhibits a change in fluorescence in the presence of double-stranded nucleic acid).

Amplification of nucleic acids may include amplification of nucleic acids or subregions of these nucleic acids. For example, amplification may include amplifying portions of nucleic acids between 50 and 300 bases long by selecting the proper primer sequences and using the PCR.

The disclosed methods may include amplifying at least one nucleic acid in the sample (preferably two nucleic acid, an more preferably three nucleic acids). Amplification mixtures may include natural nucleotides (e.g., A, C, G, T, and U) and non-natural nucleotides (e.g., iC and iG). Examples of non-natural nucleotides and bases are described in U.S. patent application publication 2002-0150900, which is incorporated herein by reference in its entirety. The nucleotides, which may include non-natural nucleotides may include a label (e.g., a quencher or a fluorophore).

The oligonucleotides of the present methods may function as primers. The oligonucleotides may include at least one non-natural nucleotide. For example, the oligonucleotides may include at least one nucleotide that is not A, C, G, T, or U (e.g., iC or iG). In some embodiments, the oligonucleotides are labeled. For example, the oligonucleotides may be labeled with a reporter that emits a detectable signal (e.g., a fluorophore); the oligonucleotides may include at least one non-natural nucleotide and a label, for example, at least one nucleotide may be labeled with a quencher (e.g., Dabcyl), and may include at least one nucleotide that is not A, C, G, T, or U (e.g., iC or iG).

As used herein, the terms “purified” or “substantially purified” refer to molecules, either nucleic or amino acid sequences, that are removed from their natural environment, isolated or separated, and are at least 60% free, preferably 75% free, and most preferably 90% free from other components with which they are naturally associated. An “isolated polynucleotide” or “isolated oligonucleotide” is therefore a substantially purified polynucleotide.

In some embodiments, the oligonucleotide may be designed not to form an intramolecular structure such as a hairpin. In other embodiments, the oligonucleotide may be designed to form an intramolecular structure such as a hairpin. For example, the oligonucleotide may be designed to form a hairpin structure that is altered after the oligonucleotide hybridizes to a target nucleic acid, and optionally, after the target nucleic acid is amplified using the oligonucleotide as a primer.

As used herein, “labels” or “reporter molecules” are chemical or biochemical moieties useful for labeling a nucleic acid (including a single nucleotide), amino acid, or antibody. “Labels” and “reporter molecules” include fluorescent agents, chemiluminescent agents, chromogenic agents, quenching agents, radionuclides, enzymes, substrates, cofactors, inhibitors, magnetic particles, and other moieties known in the art. “Labels” or “reporter molecules” are capable of generating a measurable signal and may be covalently or noncovalently joined to an oligonucleotide or nucleotide (including a non-natural nucleotide).

The oligonucleotides and nucleotides (including non-natural nucleotides) of the disclosed methods may be labeled with a “fluorescent dye” or a “fluorophore.” As used herein, a “fluorescent dye” or a “fluorophore” is a chemical group that can be excited by light to emit fluorescence. Some suitable fluorophores may be excited by light to emit phosphorescence. Dyes may include acceptor dyes that are capable of quenching a fluorescent signal from a fluorescent donor dye.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The oligonucleotides and nucleotides of the disclosed methods (including non-natural nucleotides) may be labeled with a quencher. Quenching may include dynamic quenching (e.g., by FRET), static quenching, or both.

Enzymes

Disclosed herein are methods that may utilize a polymerase, ligase and/or the polymerase chain reaction, to construct, assemble and/or amplify a large (e.g., greater than 400 nucleotides in length) double-stranded nucleic acid such as a gene or genome.

Suitable nucleic acid polymerases include, for example, polymerases capable of extending an oligonucleotide by incorporating nucleic acids complementary to a template oligonucleotide. For example, the polymerase can be a DNA polymerase.

Enzymes having polymerase activity catalyze the formation of a bond between the 3′ hydroxyl group at the growing end of a nucleic acid primer and the 5′ phosphate group of a nucleotide triphosphate. These nucleotide triphosphates are usually selected from deoxyadenosine triphosphate (A), deoxythymidine triphosphate (T), deoxycytosine triphosphate (C) and deoxyguanosine triphosphate (G). However, in at least some embodiments, polymerases useful for the methods disclosed herein also may incorporate non-natural bases using nucleotide triphosphates of those non-natural bases.

Because the relatively high temperatures necessary for strand denaturation during methods such as PCR can result in the irreversible inactivation of many nucleic acid polymerases, nucleic acid polymerase enzymes useful for performing the methods disclosed herein preferably retain sufficient polymerase activity to complete the reaction when subjected to the temperature extremes of methods such as PCR. Preferably, the nucleic acid polymerase enzymes useful for the methods disclosed herein are thermostable nucleic acid polymerases. As used herein, the term “thermostable nucleic acid polymerase” refers to an enzyme that catalyzes the polymerization of nucleosides and which is relatively stable to heat when compared, for example, to nucleotide polymerases from E. coli. Generally, the enzyme will initiate synthesis at the 3′-end of the primer annealed to the target sequence, and will proceed in the 5′-direction along the template, and if possessing a 5′ to 3′ nuclease activity, hydrolyzing an intervening, annealed oligonucleotide to release intervening nucleotide bases or oligonucleotide fragments, until synthesis terminates. A thermostable enzyme has activity at a temperature of at least about 37° C. to about 42° C., typically in the range from about 50° C. to about 75° C. Suitable thermostable nucleic acid polymerases include, but are not limited to, enzymes derived from thermophilic organisms. Examples of thermophilic organisms from which suitable thermostable nucleic acid polymerase can be derived include, but are not limited to, Thermus aquaticus, Thermus thermophilus, Thermus flavus, Thermotoga neapolitana and species of the Bacillus, Thermococcus, Sulfobus, and Pyrococcus genera. Nucleic acid polymerases can be purified directly from these thermophilic organisms. However, suitable thermostable nucleic acid polymerases, such as those described above, are commercially available.

A number of nucleic acid polymerases possess activities in addition to nucleic acid polymerase activity; these can include 5′-3′ exonuclease activity and 3′-5′ exonuclease activity. The 5′-3′ and 3′-5′ exonuclease activities are known to those of ordinary skill in the art. Nucleic acid polymerase with an attenuated 5′-3′ exonuclease activity, or in which such activity is absent, are also known in the art. In some embodiments, an exonuclease activity on a nucleic acid polymerase may be desired; in other embodiments, a nucleic acid polymerase with no exonuclease activity may be desired. Suitable nucleic acid polymerases with or without the 5′-3′ exonuclease activity are commercially available.

Polymerases can “misincorporate” bases during extension or PCR. In other words, the polymerase can incorporate a nucleotide (for example adenine) at the 3′ position on the synthesized strand that does not form canonical hydrogen base pairing with the paired nucleotide (for example, cytosine) on the template nucleic acid strand. The polymerizing or PCR conditions can be altered to decrease the occurrence of misincorporation of bases. For example, reaction conditions such as temperature, salt concentration, pH, detergent concentration, type of metal, concentration of metal, and the like can be altered to decrease the likelihood that polymerase will incorporate a base that is not complementary to the template strand. By way of example, but not by way of limitation, PCR conditions that may encourage polymerase read-through (e.g., incorporation of a natural nucleotide opposite a non-natural nucleotide) are as follows: 10 mM Bis-Tris Propane, pH 9.1; 40 mM KCl; 2 mM MgCl₂; 200 nM dNTPs, 50-200 nM amplimers, 1U. (20 μl PCR reaction) of Taq DNA polymerase or Klentaq 1 Polymerase; thermal cycling 1× 95° C. 2 min, 35× (95° C. 10 s, 58° C. 30 s, 72° C. 90 s), 1× 72° C. 5 min, soak 4° C. By way of example but not by way of limitation, PCR conditions that may discourage read-through (e.g, polymerase stall or stop): 20 mM Tris-HCl (pH 8.8), 10 mM KCl, 10 mM (NH₄)₂SO₄; 2 mM MgSO₄, 0.1% Triton® X-100, 0.1% nuclease free BSA; 2.5 U/(50 μl PCR reaction); Pfu DNA polymerase; thermal cycling 1× 95° C. 2 min; 38× (95° C. 10 s, 58° C. 10 s, 72° C. 60 s), 1× 72° C. 1 min, 4° C. soak.

As an alternative to using a single polymerase, any of the methods described herein can be performed using multiple enzymes. For example, a polymerase, such as an exo-nuclease deficient polymerase, and an exo-nuclease can be used in combination. Another example is the use of an exo-nuclease deficient polymerase and a thermostable flap endonuclease. In addition, it will be recognized that RNA can be used as a sample and that a reverse transcriptase can be used to transcribe the RNA to cDNA. The transcription can occur prior to or during PCR amplification.

Methods may also include the use of a ligase. As used herein, a ligase means an enzyme that catalyzes the joining of two strands of nucleic acid (i.e., closes nicks or discontinuities in one strand of double-stranded DNA) by creating a phosphodiester bond between the 3′ OH and the 5′ PO₄ of adjacent nucleotides. Ligases may join blunt-ended or cohesive-ended nucleic acid configurations. DNA and RNA ligases are commercially available; by way of example, but not by way of limitation, ligases may include T4 DNA ligase, T4 RNA ligase, thermostable Pfu ligase, Taq ligase.

DNA ligase(s) can act on a 5′-end overhangs (in vivo or in vitro) when a complementary or substantially complementary strand is hybridized to the overhang, even if any one of the four natural bases is “mispaired” with a non-natural nucleotide, for example, isoC (data not shown).

Oligonucleotides as PCR or Polymerase Primers

Disclosed herein are methods for the assembly, construction and/or amplification of single or double-stranded nucleic acids. Some of these methods may include the use of a polymerase to create a complement of a particular sequence; other methods may include the polymerase chain reaction to amplify a particular sequence.

In methods involving polymerases (e.g., PCR or polymerase extension), oligonucleotides of the methods can act as “primers.” These primers are designed to be complementary to sequences known to exist in a target nucleic acid to be amplified (i.e., for PCR), or to contain regions of complementarity to other oligonucleotides (i.e., to create a complement).

In PCR applications, the primers are typically chosen to be complementary to sequences that flank (and can be part of) the target nucleic acid sequence to be amplified. Preferably, the primers are chosen to be complementary to sequences that flank the target nucleic acid to be detected. Once the sequence of the target nucleic acid is known, the sequence of a primer is prepared by first determining the length or size of the target nucleic acid to be detected, determining appropriate flanking sequences that are near the 5′ and 3′ ends of the target nucleic acid sequence or close to the 5′ and 3′ ends, and determining the complementary nucleic acid sequence to the flanking areas of the target nucleic acid sequence using standard Watson-Crick base pairing rules, and then synthesizing the determined primer sequences.

For oligonucleotides designed to contain regions complementary to other oligonucleotides, regions of complementarity are generally designed into the 5′ and the 3′ ends of the complementary oligonucleotides. For example, 10 bases at the 5′ end of a first oligonucleotide may be complementary to 10 bases at the 3′ end of a second oligonucleotide. In this example, the first oligonucleotide is the “template” or “target” and the second oligonucleotide is the “primer.”

The preparation of oligonucleotides as primers can be accomplished using any suitable methods known in the art, for example, cloning and restriction of appropriate sequences and direct chemical synthesis. Chemical synthesis methods can include, for example, the phosphotriester method described by Narang et al. Methods in Enzymology 68:90 (1979), the phosphodiester method disclosed by Brown et al. Methods in Enzymology 68:109 (1979), the diethylphosphoramidate method disclosed in Beaucage et al. Tetrahedron Letters 22:1859 (1981), and the solid support method disclosed in U.S. Pat. No. 4,458,066, all of which are incorporated herein by reference.

The ability of the first primer and second primer or a first oligonucleotide (template) and a second oligonucleotide (primer) to form sufficiently stable hybrids depends upon several factors, for example, the degree of complementarity exhibited between the primer and the target or template nucleic acid. Typically, an oligonucleotide having a higher degree of complementarity to its target will form a more stable hybrid with the target.

Additionally, the length of the primer or length of the region of complementarity can affect the temperature at which the primer will hybridize to the target or template nucleic acid. Generally, a longer primer or complementary region will form a sufficiently stable hybrid to the target nucleic acid sequence at a higher temperature than will a shorter primer or complementary region.

Further, the presence of high proportion of G or C or of particular non-natural bases in the primer or complementary regions can enhance the stability of a hybrid formation. This increased stability can be due to, for example, the presence of three hydrogen bonds in a G-C interaction or other non-natural base pair interaction compared to two hydrogen bonds in an A-T interaction.

Stability of a nucleic acid duplex can be estimated or represented by the melting temperature, or “Tₘ.” The Tₘ of a particular nucleic acid duplex under specified conditions is the temperature at which 50% of the population of the nucleic acid duplexes dissociate into single-stranded nucleic acid molecules. The Tₘ of a particular nucleic acid duplex can be predicted by any suitable method. Suitable methods for determining the Tₘ of a particular nucleic acid duplex include, for example, software programs. Primers suitable for use in the methods disclosed herein can be predetermined based on the predicted Tₘ of an oligonucleotide duplex that comprises the primer.

In a PCR reaction, when the first primer and second primer are annealed to the target nucleic acid, a gap exists between the 3′ terminal nucleotide of the first primer and the 3′ terminal nucleotide of the second primer. The gap comprises a number of nucleotides of the target nucleic acid. The gap can be any number of nucleotides provided that the polymerase can effectively incorporate nucleotides into an elongating strand to fill the gap during a round of the PCR reaction (e.g., a round of annealing, extension, denaturation). Typically, a polymerase can place about 30 to about 100 bases per second. Thus, the maximum length of the gap between primers depends upon the amount of time within a round of PCR where the temperature is in a range in which the polymerase is active and the primers are annealed.

Similarly, when a “primer oligonucleotide” anneals to a “template oligonucleotide” the polymerase is used to create a second strand or complement to the template oligonucleotide. Considerations regarding stability of the duplex or complementary regions, including length, G:C content, Tm, and time of the reaction may also be considered.

Non-Natural Bases

As contemplated in the methods disclosed herein, oligonucleotides typically comprises at least one non-natural base. DNA and RNA are oligonucleotides that include deoxyriboses or riboses, respectively, coupled by phosphodiester bonds. Each deoxyribose or ribose includes a base coupled to a sugar. The bases incorporated in naturally-occurring DNA and RNA are adenosine (A), guanosine (G), thymidine (T), cytosine (C), and uridine (U). These five bases are “natural bases”. According to the rules of base pairing elaborated by Watson and Crick, the natural bases can hybridize to form purine-pyrimidine base pairs, where G pairs with C and A pairs with T or U. These pairing rules facilitate specific hybridization of an oligonucleotide with a complementary oligonucleotide.

The formation of these base pairs by the natural bases is facilitated by the generation of two or three hydrogen bonds between the two bases of each base pair. Each of the bases includes two or three hydrogen bond donor(s) and hydrogen bond acceptor(s). The hydrogen bonds of the base pair are each formed by the interaction of at least one hydrogen bond donor on one base with a hydrogen bond acceptor on the other base. Hydrogen bond donors include, for example, heteroatoms (e.g., oxygen or nitrogen) that have at least one attached hydrogen. Hydrogen bond acceptors include, for example, heteroatoms (e.g., oxygen or nitrogen) that have a lone pair of electrons.

The natural bases, A, G, C, T, and U, can be derivatized by substitution at non-hydrogen bonding sites to form modified natural bases. For example, a natural base can be derivatized for attachment to a support by coupling a reactive functional group (for example, thiol, hydrazine, alcohol, amine, and the like) to a non-hydrogen bonding atom of the base. Other possible substituents include, for example, biotin, digoxigenin, fluorescent groups, alkyl groups (e.g., methyl or ethyl), and the like.

Non-natural bases, which form hydrogen-bonding base pairs, can also be constructed as described, for example, in U.S. Pat. Nos. 5,432,272; 5,965,364; 6,001,983; 6,037,120; 6,140,496; U.S. published application no. 2002/0150900; all of which are incorporated herein by reference. Suitable bases and their corresponding base pairs may include the following bases in base pair combinations (iso-C/iso-G, K/X, H/J, and M/N):

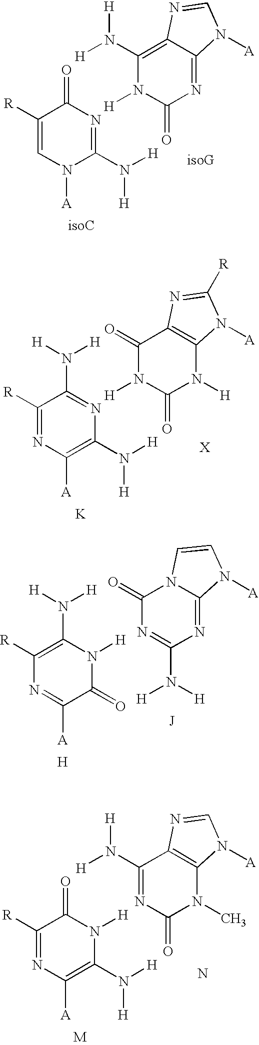

where A is the point of attachment to the sugar or other portion of the polymeric backbone and R is H or a substituted or unsubstituted alkyl group. It will be recognized that other non-natural bases utilizing hydrogen bonding can be prepared, as well as modifications of the above-identified non-natural bases by incorporation of functional groups at the non-hydrogen bonding atoms of the bases.

The hydrogen bonding of these non-natural base pairs is similar to those of the natural bases where two or three hydrogen bonds are formed between hydrogen bond acceptors and hydrogen bond donors of the pairing non-natural bases. One of the differences between the natural bases and these non-natural bases is the number and position of hydrogen bond acceptors and hydrogen bond donors. For example, cytosine can be considered a donor/acceptor/acceptor base with guanine being the complementary acceptor/donor/donor base. Iso-C is an acceptor/acceptor/donor base and iso-G is the complementary donor/donor/acceptor base, as illustrated in U.S. Pat. No. 6,037,120, incorporated herein by reference.

Other non-natural bases for use in oligonucleotides include, for example, naphthalene, phenanthrene, and pyrene derivatives as discussed, for example, in Ren et al., J. Am. Chem. Soc. 118, 1671 (1996); McMinn et al., J. Am. Chem. Soc. 121, 11585 (1999); Ohtsuki, et al., PNAS 98(9):4922-4925 (2001); Gao et al, Agnew. Chem. Int. Ed. 44:3118-3122 (2005), all of which are incorporated herein by reference. These bases do not utilize hydrogen bonding for stabilization, but instead rely on hydrophobic or van der Waals interactions to form base pairs.

Non-natural bases can be recognized by many enzymes that catalyze reactions associated with nucleic acids. While a polymerase requires a complementary nucleotide to continue polymerizing an extending oligonucleotide chain, other enzymes do not require a complementary nucleotide. If a non-natural base is present in the template and its complementary non-natural base is not present in the reaction mix, a polymerase will typically stall (or, in some instances, misincorporate a base when given a sufficient amount of time) when attempting to extend an elongating primer past the non-natural base. However, other enzymes that catalyze reactions associated with nucleic acids, such as ligases, kinases, nucleases, polymerases, topoisomerases, helicases, and the like can catalyze reactions involving non-natural bases. Such features of non-natural bases can be taken advantage of, and are within the scope of the presently disclosed methods and kits.

For example, non-natural bases can be used to generate duplexed nucleic acid sequences having a single strand overhang. This can be accomplished by performing a PCR reaction on a target nucleic acid in a sample, the target nucleic acid having a first portion and a second portion, where the reaction system includes all four naturally occurring dNTP's, a first primer that is complementary to the first portion of the target nucleic acid, a second primer having a first region and a second region, the first region being complementary to the first portion of the target nucleic acid, and the second region being noncomplementary to the target nucleic acid. The second region of the second primer comprises a non-natural base. The first primer and the first region of the second primer hybridize to the target nucleic acid, if present. Several rounds of PCR will produce an amplification product containing (i) a double-stranded region and (ii) a single-stranded region. The double-stranded region is formed through extension of the first and second primers during PCR. The single-stranded region includes the one or more non-natural bases. The single-stranded region of the amplification product results because the polymerase is not able to form an extension product by polymerization beyond the non-natural base in the absence of the nucleotide triphosphate of the complementary non-natural base. In this way, the non-natural base functions to maintain a single-stranded region of the amplification product.

As mentioned above, the polymerase can, in some instances, misincorporate a base opposite a non-natural base. The misincorporation may take place because the reaction mix does not include a complementary non-natural base. Therefore, if given sufficient amount of time, the polymerase can, in some cases, misincorporate a base that is present in the reaction mixture opposite the non-natural base.

For purposes of this description and by way of example but not by way of limitation, a gene assembly methods based on ligation and a gene assembly method described as two-step PCR method will be described. It is understood that one skilled in the art could use the methods and techniques disclosed herein with modifications of the described methods, or with any of a variety of gene assembly methods.

Exemplary Gene Assembly Methods Using Non-Natural Nucleotides

As a first step, a plurality of oligonucleotides representing the entire, double-stranded sequence of interest are synthesized such that each oligonucleotide has a region of complementarity to one or two different oligonucleotides.

For example, oligonucleotide A is 60 nucleotides long, and is designed such that the 30 most 5′ nucleotides are fully complementary to the 30 most 3′ nucleotides of oligonucleotide B, which is also a total of 60 nucleotides long. Oligonucleotide C, also 60 nucleotides long, has 30 nucleotides on its 3′ end that are complementary to the 30 nucleotides on the 5′ end of oligonucleotide B. Thus, oligonucleotide B has regions of complementarity and can hybridize to both oligonucleotide A and oligonucleotide C. Oligonucleotides D and E may hybridize to the “overhangs” remaining after the hybridization of A, B and C. The 3′ and the 5′ most terminal oligonucleotides used to form the sequence of interest need only have a region of complementarity to one other oligonucleotide. In this way, a full-length, single or double stranded oligonucleotide is assembled. The oligonucleotides may be any convenient length. Likewise the regions of complementarity between oligonucleotides may be any convenient length. Computer programs, software, algorithms and the like to aid in the design of oligonucleotides for optimal hybridization reactions are know to those of skill in the art. Additionally, regions of complementarity may be fully complementary or may be partially complementary.

Annealing a plurality of complementary oligonucleotides in the manner described above results in a double-stranded nucleic acid molecule with nicks in the sugar-phosphate backbone. A means to couple adjacent oligonucleotides is then used. For example, a ligase, such as DNA ligase, may be used to create a phosphodiester bond between the 3′ OH and the 5′ PO₄ of neighboring oligonucleotides. In the above example, a ligase would seal the nick between 3′ end of oligonucleotides C and the 5′ end of oligonucleotide A. Any convenient means of coupling (e.g., chemical or enzymatic) may be used. The single or double-stranded sequence can then be amplified using PCR primers specific for the full-length sequence.

To increase the specificity of the hybridization reaction, oligonucleotides are synthesized with complementary regions containing one or more complementary non-natural nucleotides, for example, iC and iG.

The PCR reaction may be performed using all natural nucleotides, or may be performed using both natural and non-natural nucleotides.

Another gene synthesis method involves assembling oligonucleotides on a single-stranded full-length template. The oligonucleotides, which are complementary to different regions of the template, are synthesized containing one or more non-natural nucleotides. The oligonucleotides hybridize to the template, and a coupling means, such as a ligase may then be used to covalently join neighboring oligonucleotides. The newly formed double-stranded molecule may then be denatured and the covalently coupled single strand may be amplified by methods known in the art. The amplification reaction may include natural bases, or the amplification reaction may include natural and non-natural bases. Or, the newly formed double-stranded molecule may be denatured and the covalently coupled single strand may be hybridized to a second plurality of oligonucleotides containing at least one non-natural nucleotide complementary to non-natural nucleotides in the covalently coupled single strand. The hybridized second plurality of oligonucleotides may then be covalently coupled, the two strands denatured and the process repeated with a third and fourth plurality of oligonucleotides, or the denatured strands may be amplified. Amplification may be in the presence of natural or a combination of natural and non-natural nucleotides.

Exemplary Two-Step Gene Synthesis Methods Using Non-Natural Nucleotides

In the first step, multiple oligonucleotides containing overlapping complementary regions are allowed to anneal under optimized hybridization conditions. As described above, optimization may involve an evaluation of the length of complementary regions of the oligonucleotides, the sequence of the complementary regions including G:C content, the Tm of the annealed hybrids, the overall length of the oligonucleotides and other factors known in the art. Computer programs and software to aid in the design of such oligonucleotides are also known in the art (see, e.g., Hoover and Lubkowski, Nucleic Acids Res. 30(10):e43 (2001); Rydzanick et al., Nucleic Acids Res. 33:W521-525 (2005)).

The oligonucleotides are designed to overlap, such that a properly and fully annealed set of oligonucleotides yields a precursor, full-length nucleic acid that is representative of the sequence of interest. This precursor full-length sequence contains both double-stranded regions (in the regions of complementarity between oligonucleotides) and single-stranded regions. In preferred embodiments, the regions of complementarity between oligonucleotides are located in the 5′ and the 3′ regions of the oligonucleotides. For example, the 10 most 5′ nucleotides of oligonucleotide A are complementary to the 10 most 3′ nucleotides of oligonucleotide B. Regions such as this 10-base overlap create the double-stranded regions in the properly and fully annealed precursor, full-length sequence.

The oligonucleotides are also designed such that each oligonucleotide may act a “primer” for the synthesis of a complement to the oligonucleotide to which it anneals (the “template” oligonucleotide). For example, oligonucleotide A, described above is the “template” oligonucleotide, and oligonucleotide B is the “primer” oligonucleotide. Oligonucleotide B is the “primer” because the 3′ end of oligonucleotide B is available for priming by a polymerase using oligonucleotide A as the template. A polymerase is then used to extend oligonucleotide B, generating a complement to the oligonucleotide A template. A complement therefore, comprises the primer oligonucleotide sequence and the complementary sequence of the single-stranded “template” region of the template oligonucleotide (the region of the template oligonucleotide not covered by the primer oligonucleotide).

A single oligonucleotide may act as both a primer and as a template. For example, continuing with the example above, a third oligonucleotide C contains 10 nucleotides at its 5′ end that are complementary to 10 nucleotides at the 3′ end of oligonucleotide A. Thus, oligonucleotide A will be a primer for oligonucleotide C, the template, and a polymerase will create a complement that includes the primer A sequence as well as the complementary sequence of the single-stranded region of template oligonucleotide C.

Thus, when polymerase and the proper reactants (dNTP's, buffer, etc.) are combined with the properly annealed oligonucleotides, the polymerase reaction effectively “fills in” the single stranded gaps between the double-stranded complementary regions. This results in full-length, double-stranded molecule. Nicks in the strand backbones may be repaired (e.g., adjacent complements may be coupled) by, chemical or enzymatic coupling methods, such as for example, a ligase.

The oligonucleotides in a singe reaction tube can be either for the entire desired sequence (as described above), or for portions or blocks of the desired sequence. For example, oligonucleotides coding for the first ⅓ of the desired sequence are placed in reaction tube A, oligonucleotides coding for the second ⅓ of the desired sequence are placed in reaction tube B, and oligonucleotides coding for the last ⅓ of the desired sequence are placed in reaction tube C. The oligonucleotides in the individual reactions are allowed to anneal, and polymerase reactions are performed to create the complementary strands. Then, the separate reactions may be combined and allowed to anneal to form the final, full-length product, or a PCR reaction may be performed to enrich for each individual “full-length” (e.g., ⅓-length) product in each reaction. This PCR reaction may be done by using primers specific for each individual “full-length” (e.g., ⅓-length) product. These PCR products may then be combined to form the final full-length sequence.

Because a range of products of different lengths result from the different possible combinations of annealing that involve less than all the oligonucleotides, the second step of the two-step reaction involves PCR. In the second step, primers specific for the 3′ end and the 5′ end of the final full-length sequence are used to amplify and enrich for the full-length product. The amplified, full-length product can be further purified (e.g., by gel purification), cloned into an appropriate vector and sequenced to check for any errors in oligonucleotide synthesis or assembly and PCR. Finally, full-length products may be tested and used in a variety of biological systems. For example, the full-length product may be transfected into a cell or an organism and replicated or expressed.

In some methods, the regions of complementarity or overlaps between the oligonucleotides can be artificially created. For example, DNA oligomers, small single-stranded sequences, can be added (e.g., ligated) onto a plurality of oligonucleotides. The oligonucleotides contain sequence representative of the gene or sequence of interest, while the oligomer sequences are complementary to each other. For example, a set of oligomers, set A, may be complementary to another set of oligomers, set B, while set C is complementary to set D, etc. In preferred embodiments, the oligomers contain at least one non-natural nucleotide such as iC or iG, and complementary oligomers contain at least one non-natural, complementary base-pair. Any number of complementary sets of oligomers may be used. The oligomers may be ligated to the oligonucleotides by methods known in the art to create “tagged oligonucleotides.” The tagged oligonucleotides are then allowed to hybridize via complementary oligomer regions. Similar to the oligonucleotides described in the methods above, the tagged oligonucleotides can act as both primers and templates for the synthesis of complements. For example, tagged oligonucleotide A is made up of oligonucleotide A and oligomer A. During the ligation reaction, oligomer A was ligated to the 5′ end of oligonucleotide A. Tagged oligonucleotide B is made up of oligonucleotide B and oligomer B. During the ligation reaction, oligomer B was ligated to the 3′ end of oligonucleotide B. Oligomer A and oligomer B are complementary and allow tagged oligonucleotides A and B to anneal. Tagged oligonucleotide B can act as a primer and tagged oligonucleotide A can act as template in a polymerase reaction to synthesize a complement. Here, the complement would contain the sequence of oligonucleotide B, including the oligomer B sequences, and the single-stranded region of tagged oligonucleotide A. By building tagged oligonucleotide chains, a full-length double-stranded molecule can be constructed. The full-length molecule can then be amplified by PCR, cloned, sequenced and expressed as previously described.

Even though oligonucleotide sequences used for assembly can be designed to have a uniform annealing temperature and can be checked for regions of overlap outside of the desired priming regions necessary for assembly, non-specific hybridizations still occur. This is especially problematic when many overlapping oligonucleotides are used to make the full-length sequence. To improve annealing specificity and molecular recognition between complementary oligonucleotide regions, one or more non-natural nucleotide can be incorporated into the oligonucleotide sequences. The addition of non-natural nucleotides effectively increase the number of possible base pairs that can form between oligonucleotides from two (A:T in DNA or A:U in RNA and G:C) to three or more.

In preferred embodiments, the non-natural nucleotides are incorporated into the complementary regions of the oligonucleotides in the 5′ and 3′ ends. In more preferred embodiments, the non-natural nucleotide is the terminal 5′ or 3′ base. For example, an oligonucleotide A contains the non-natural nucleotide (for example, iC) as a 5′ terminal nucleotide, and also contains another iC five nucleotides distant from the terminal iC. The 10 most 5′-nucleotides of oligonucleotide A are complementary to the 10 most 3′ nucleotides of another oligonucleotide B. Oligonucleotide B contains a non-natural nucleotide (iG) at its terminal 3′ end, and also contains another iG five nucleotides distant from the 3′ terminal iG. Once hybridized, oligonucleotide A is the template, and oligonucleotide B is the primer for any subsequent PCR or polymerase reactions. Oligonucleotides A and B may also contain non-natural bases in their opposite ends (the 3′ end for A and the 5′ end for B) and may act as primer and template for additional oligonucleotides, C and D.

The polymerase reaction may be performed with all natural nucleotides or a combination of natural and non-natural nucleotides. For example, oligonucleotides may be designed to contain non-natural nucleotides in the “non-complementary” regions (i.e., the “template” region). In the presence of non-natural, complementary mononucleotides, the polymerase will incorporate the non-natural complement into the growing oligonucleotide chain. Alternatively, in the presence of only natural bases and under the proper reaction conditions, the polymerase may incorporate a natural base opposite the non-natural base.

Similarly, the PCR reaction to enrich for the full-length product may also be performed with all natural or a combination of natural and non-natural nucleotides. The PCR reaction may be used to effectively “remove” the non-natural bases. For example, in the presence of only natural bases and under the proper conditions (e.g., temperature, pH, enzyme, etc.), the polymerase in the PCR reaction may incorporate a natural base as a complement to a non-natural base.

Proximity Effects

Another method to improve annealing specificity and molecular recognition between complementary oligonucleotides takes advantage of proximity effects. Proximity effects have been noted in many systems. For example, when an intermolecular reaction is replaced by an intramolecular reaction, a rate increase is noted. Likewise, when an enzyme positions a substrate near an active site, or when a catalyst positions substrate near a catalytic group, proximity effects can be important.

Thus, oligonucleotides that are to designed to anneal to each other in an assembly reaction (e.g., primer/template oligonucleotides) are more likely to hybridize quickly and properly if they are closer together in space. One means of spatially manipulating oligonucleotides is to specifically position them on a solid support such that, for example, primers are adjacent to their templates. Oligonucleotide positioning may be accomplished by methods known in the art, including, for example, spotting, printing or actually synthesizing the oligonucleotides directly on the support. Supports may include for example, glass, silicon chips or nylon membranes. Oligonucleotides may be reversibly or irreversibly immobilized on the solid support, or the oligonucleotides may be covalently bound to the support.

For example, A DNA microarray is made up of a plurality of sets of single stranded DNA oligonucleotides. The oligonucleotides in a given set are identical in sequences but different from the oligonucleotide sequence of other sets. The oligonucleotide sets are made on the microarray and designed such that at least one region of each oligonucleotide is complementary to a region on at least one other oligonucleotide set on the array. Oligonucleotide sets with complementary regions may be located on the array at proximal positions, optionally at a distance of no more than about 10 microns. Accordingly, when the oligonucleotides are released from the substrate, oligonucleotides with complementary regions will be in close proximity, and proper annealing to generate the full-length representative sequence will be more efficient, more reproducible and accomplished with fewer assembly errors.

The oligonucleotides of a particular set or sets may remain bound to the substrate while others are released. Alternatively all of the oligonucleotides may be released from the substrate and allowed to anneal and self-assemble.

Repair/Conversion of Base-Pairs Comprising Non-Natural Bases

A method of repairing a DNA molecule with base non-natural bases or with mismatched base pair is described. Mismatches involving a non-natural base of most any sequence composition can be generated. The method entails using a cell's repair machinery to correct the mismatch and convert the base pair comprising at least one non-natural base into a natural base pair.

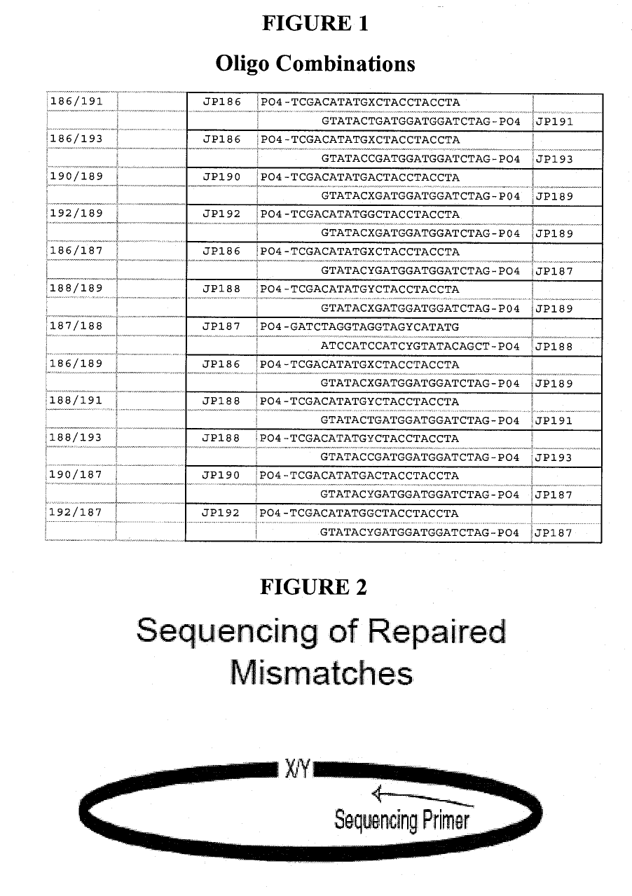

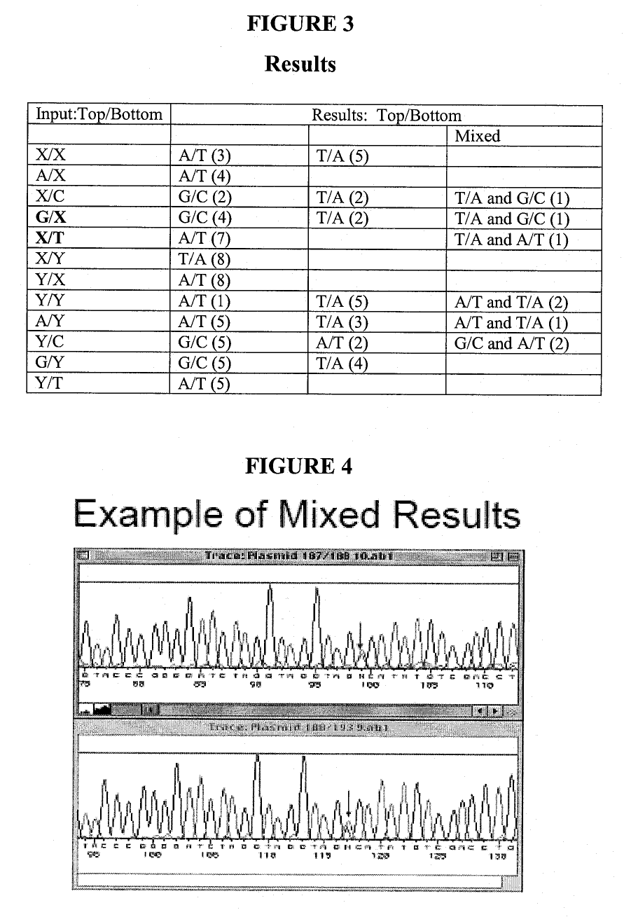

Oligonucleotides containing different mismatches were designed (FIG. 1) and ligated into pUC18. In the example, “X” corresponds to iC and “Y” corresponds to iG (described above). NovaBlue cells were transformed, and after growth, minipreps were done on white colonies. The resulting repairs were analyzed by sequencing using the BigDye terminator kit. FIG. 2 shows a scheme for sequencing to detect the result of the DNA repair/conversion. FIG. 3 shows the results of the conversion. The input sequence indicates the base pair mismatch that was introduced into the NovaBlue cells. The results column indicates the converted sequence with the number of colonies having that conversion indicated in parentheses. Colonies having mixed results were determined by examining the sequencing traces and observing two peaks at a given nucleotide position (FIG. 4). The results indicate that a DNA sequence having a mismatched base pair, wherein one of the bases is a non-natural base, can be converted to a natural base pair according to the method described here.

Cloning

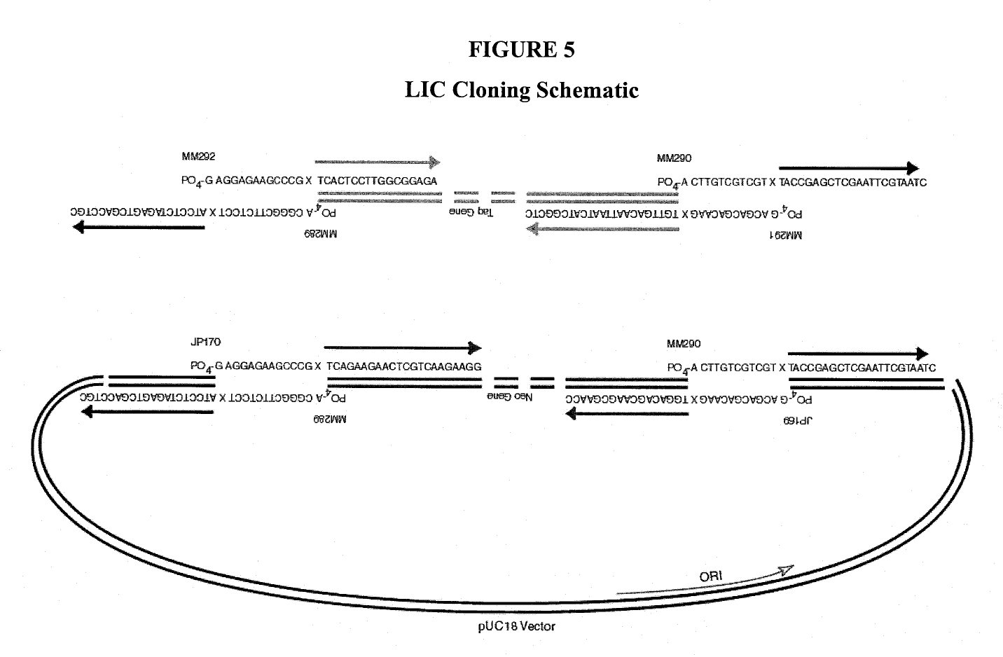

A method of generating DNA fragments with single-stranded, ligase-ready overhangs during PCR is described. Overhangs of most any sequence composition and length can be generated during PCR by simply adding a single non-standard base into the PCR primer. The method generates polymerase chain reaction products composed of double-stranded (ds) DNA flanked by single-stranded (ss) DNA or RNA overhangs (FIG. 5). The method entails using PCR primers containing non-standard bases which cannot be copied by certain thermostable DNA polymerases. When the complementary non-standard base triphosphate is not supplied during the PCR reaction, the overhangs result. The resulting amplicons can be used for directional cloning or solid phase ligation. Possible advantages of this method includes control over both the length and sequence of the overhangs, and elimination of the need for additional enzymes as tools for gene engineering. One method involves placing a single iso-C at the site where the overhang is to begin.

### EXAMPLE 1

### Ligation Dependent Cloning

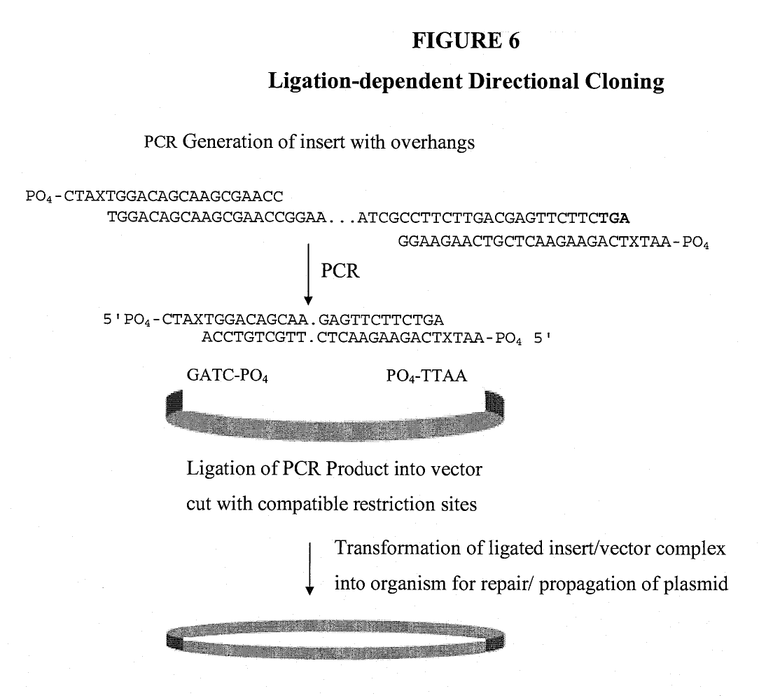

According to the described methods, ligation-dependent directional cloning of a kanamycin resistance gene was performed (FIG. 6). The insert was generated using PCR amplification of the Neo gene and promoter from pCR4TOPO plasmid using the following primers:: JP165: PO₄—CTAXTGGACAGCAAGCGAACC and JP166: PO₄-AATXTCAGAAGAACTCGTCAAGAAGG. As a positive control, the same sequence was amplified using primers containing EcoRI and XbaI sites: JP152: PO₄-GCTCTAGATGGACAGCAAGCGAACC and JP155: PO₄-GGAATTCTCAGAAGAACTCGTCAAGAAGG. The following enzymes were used: Stratagene cloned Pfu/1×Pfu buffer; Roche Pwo/1×Pwo buffer; Epicentre Tfl/10 mM BTP pH 9.1, 40 mM Kac, 2 mM MgCl₂; Epicentre Tfl/1×Tfl buffer (1); Tth/1×Tth buffer (2); Klentaq/10 mM BTP pH 9.1, 40 mM Kac, 2 mM MgCl₂; Amplitaq/10 mM BTP pH 9.1, 40 mM Kac, 2 mM MgCl₂; (Note: Amplitaq also used to amplify 152/155 control insert). Other reagents included (at final concentration: 200 mM dNTPs, 0.5 μM primers, 1 amol template/rxn. Amplification for Tfl, Tth, Amplitaq, and Klentaq polymerase was conducted at: 95° C., 2 min; then 38 cycles of 95° C., 10 sec; 58° C., 30 sec; 72° C., 30 sec. Amplification for Pfu and Pwo polymerases was conducted at: 95° C., 2 min; then 38 cycles of 95° C., 20 sec; 58° C., 5 sec; 72° C., 60 sec.

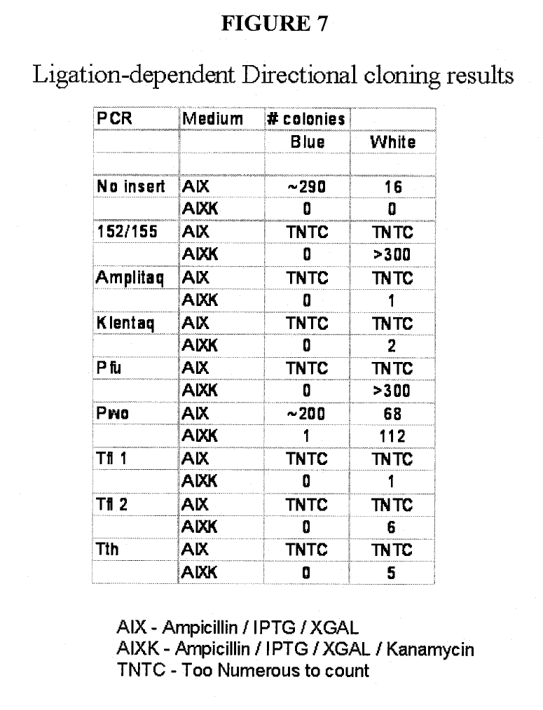

Following amplification, the excess PCR primers, dNTPs, and enzyme were removed using BM High Pure PCR kit. To prepare the vector, approximately 10 μg of pUC18 was digested using 80 U EcoRI and 80 U XbaI at 37° C. overnight in 200 μl 1×NEB2 buffer. This reaction was then purified with the BM High Pure PCR purification kit and eluted in 100 μl Tris pH 8.5. This purification method was suitable due to the short length of fragment removed. Similarly the JP 152/155 insert PCR (+control) was prepared by digesting approximately 30 μl of PCR product using 40 U EcoRI and 40 U XbaI at 37° C. overnight in 100 μl 1×NEB2 buffer. This reaction was then purified on BM High Pure PCR purification kit and eluted in 75 μl Tris pH 8.5. This purification method was suitable due to the short lengths of fragments removed. For the ligation reactions, 5 μl each PCR was added to 1 μl 10×T4 ligase buffer, 1 μl 3 U/μl T4 DNA ligase, and 300 ng EcoRI/XbaI cut pUC18 in 10 μl final volume. Reactions were incubated 4° C. 15 hrs, 10° C. 15 hrs, and 15° C. 15 hrs, then were heated to 65° C. 20 min to inactivate ligase, followed by transformation of 5 μl of the ligation reaction into 50 μl competent TOP10 cells. Plates were incubated 37° C. overnight, and colonies were counted. The results are shown in FIG. 7.

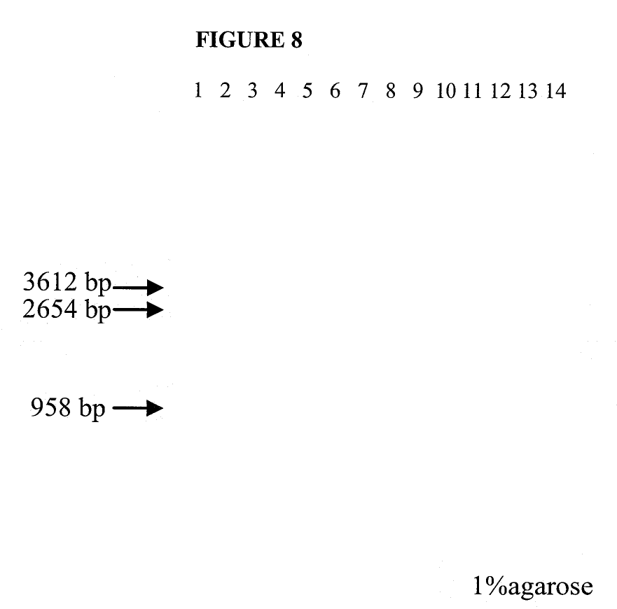

The directionality of the cloned fragments was verified. Twelve isolated colonies were chosen from ligation-dependent experiment and plasmids were prepared. Colonies chosen included: Pfu white 1-3; Pwo white 1-2; Tth white 1-2; Tfl white 1-2; Amplitaq 1; JP152-155 white 1 (+ control white). It was determined that plasmids containing incorrect orientation of insert will result in 3139 bp and 473 bp bands when digested with Nco1 and Nde1, and plasmids with correct orientation inserts will result in 2654 bp and 958 bp fragments. Restriction digests were performed using 20 U of each restriction endonuclease, approximately 0.5 μg plasmid DNA in 50 μl. The results are shown in FIG. 8 (Lane: 1: 1 kb DNA ladder; Lane 2: Pfu1 Nco1/Nde1; Lane 3: Pfu1 Nde1 only; Lane 4: Pfu1 Nco1 only; Lane 5: Pfu2 Nco1/Nde1; Lane 6: Pfu3 Nco1/Nde1; Lane 7: Pwo1 Nco1/Nde1; Lane 8: Pwo2 Nco1/Nde1; Lane 9: Tth1 Nco; Lane 10: Tth2 Nco1/Nde; Lane 11: Tfl1 Nco1/Nde1; Lane 12: Tfl2 Nco1/Nde; Lane 13: Amplitaq Nco1/Nde1; Lane 14 JP152/155 Nco1/Nde1.

### EXAMPLE 2

### Ligation Independent Cloning

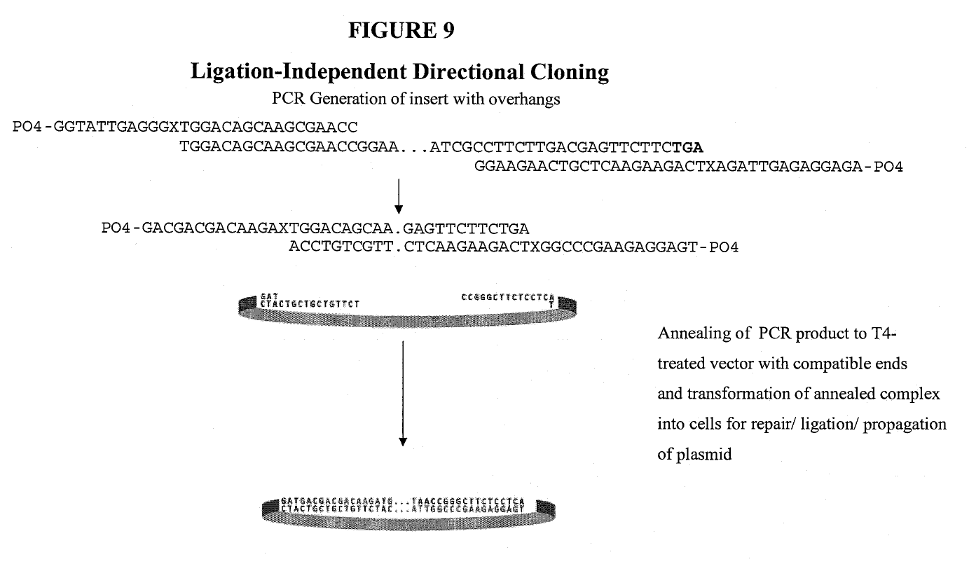

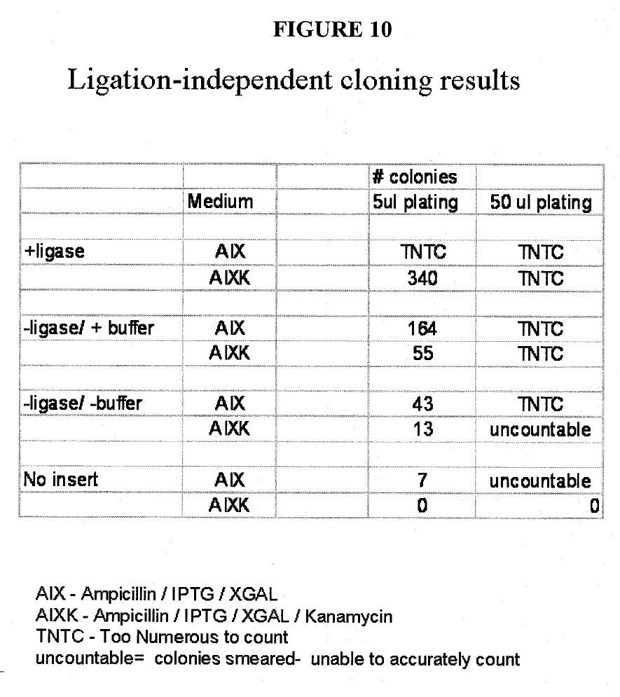

According to the described methods, ligation-independent directional cloning of a kanamycin resistance gene was performed (FIG. 9). The insert was generated using PCR amplification of the Neo gene and promoter were from pCR4TOPO plasmid using the following primers: JP169: PO₄-GGTATTGAGGGXTGGACAGCAAGCGAACC and JP170: PO₄-AGAGGAGAGTTAGAXTCAGAAGAACTCGTCAAGAAGG. 50 μl PCR reactions each contained: 2.5 U Pfu DNA polymerase; 0.5 μM JP169/170; 200 mM dNTPs; ˜1 amol pCR4TOPO. Cycling was conducted at 95° C., 2 min, then 38 cycles of 95° C., 10 sec; 58° C., 5 sec; 72° C., 1 min. The PCR products were treated with BM High Pure PCR purification kit, eluted in H₂O, and adjusted to 50 mM Tris pH 8, 10 mM MgCl₂, 1 mM rATP. Annealing reactions were performed with or without T4 ligase, according to standard conditions. All reactions were incubated at room temp for 5 min. Then 1 μl 25 mM EDTA was added and incubated 5 min, followed by transformation into NovaBlue competent cells. The cells were plated onto Amp IPTG XGAL, and Amp IPTG Xgal Kan and incubated at 37° C. overnight. Colonies were counted. The results are shown in FIG. 10. The results show that the described methods could result in successful cloning of an insert, even in the absence of a ligation step.

### EXAMPLE 3

One of the ROC experiments that we performed was to generate an “ABC” chimera through separate amplification of the “A” (the hGluR2Flop exon), “B” (intron 1 from the human β-globin gene), and “C” (the hGluR2Flip exon) fragments, followed by ligation of the amplified products.

When introduced into competent cells, longer overhangs (e.g., 13 nucleotides) can be ligated into the appropriate vectors by the endogenous ligases, negating the need for a prior ligation step.

The overhangs can be used to recombine DNA fragments at most any sequence location, creating chimeric genes composed of DNA fragments that have been joined without the insertion, deletion, or alteration of even a single base pair.

To create 5′ overhangs using these chimeric primers in the amplification reactions, a thermostable DNA polymerase that did not copy RNA was used. Vent polymerases were reported not to have such activity (unless Mn²⁺ was added to the reaction buffer). Therefore, a polymerase such as Vent or Vent exo(−) may be used.

The simplest test for the presence of the expected 5′ overhangs was to perform a ligation reaction and ask whether a chimeric gene of the appropriate sequence was generated. Each of the parental amplification products were combined in approximately equimolar amounts in a ligation reaction. A chimeric product of appropriate size would be 360 bp. The ligation mixture was amplified using primers (5′-Flop and 3′-Flip) that would flank the expected chimera. Any of the desired ligated chimeric products would be DNA-RNA hybrid molecules containing RNA nucleotides at both ligation junctions. Such hybrid chimeras could only be amplified with a DNA polymerase that was capable of reading through these RNA junction points. Taq polymerase was used; however, Taq or Tth DNA polymerase may be used. Taq polymerase generated a product of the expected size (360 bp). This product was cloned and sequenced. Results indicate that both ligation junctions had a single copy of the expected junction sequence in all 11 clones sequenced.

DNA-Overhang Cloning (DOC)

The rationale of the DOC method is to use primers containing a stretch of nucleotides that can be removed from the amplification product. Since Pfu does not copy RNA (according to Stratagene product literature), PCR initially generated products with 5′-RNA overhangs. These products were filled in using Tth polymerase, so that blunt-ended products were produced. Pfu was chosen due to its reported high fidelity. The blunt-ended products were converted to products containing 3′-DNA overhangs by removing the ribonucleotides through exposure to mild base. This treatment hydrolyzes the backbone phosphodiester bonds of the RNA, leaving a 3′-phosphate and a 5′-hydroxyl.

To ligate the product molecules, they were first treated with kinase. The phosphorylated products were then ligated, and tested for proper chimera size (360 bp) by amplification in a PCR reaction using the 5′-Flop and 3′-Flip primers and Pfu polymerase. Amplification products of the expected size were observed in both the parental and chimeric amplification reactions performed in this experiment (data not shown). The chimeric product was cloned and sequenced. The sequences of both ligation junctions of Flop-β-Flip were correct in six of eight clones that were sequenced. Two clones each had an error at one of the ligation sites. This may be due to Tth polymerase introduced errors during the fill-in step of the procedure.

In a separate experiment, we found that the products of a DOC ligation reaction could be cloned directly into a vector for replication in bacteria without a chimeric amplification step. As described above, chimeric primers were designed that, when used in a DOC experiment, generate Flop, intron 1, and Flip PCR products that could be ligated directionally. In addition, the primers were designed such that NaOH treatment of the PCR products creates an upstream overhang on the Flop exon that is compatible with an ApaI overhang, and a downstream overhang on the Flip exon that is compatible with a Pst I overhang. All three fragments were incubated together in the presence of ligase and pBluescript II SK (−) that had been digested with ApaI and PstI. An aliquot of the ligation mixture was transformed directly into Escherichia coli, and the expected chimeric clone was readily isolated, sequenced, and found to be perfect (data not shown).

To test the generality of this approach, we used DOC to generate an additional eight seamless chimeric genes ranging in size from 643 bp to 2.9 kb (data not shown). All eight chimeras were generated by directional three-molecule ligation. These chimeras were generated using M-MLV reverse transcriptase (RT), rather than Tth, to fill in 5′-RNA overhangs. When M-MLV RT was used, no errors were detected at any of the ligation points.

Experimental Protocol

PCR amplification generating products with 5′-RNA overhangs: Parental PCR reactions. Each 100 μl reaction contained 2 U of Vent exo(−) polymerase (New England Biolabs, NEB; Beverly, Mass.), 1× Thermopol buffer (10 mM KCl, 10 mM (NH₄)₂SO₄, 20 mM Tris, 2 mM MgSO₄, 0.1% Triton X-100), 200 μM dNTPs, 5 ng of template DNA (GluR-B #7 for Flop and Flip, H β T7 for β-globin intron 1) and phosphorylated primers (from Primer set 1) at a final concentration of 0.4 μM each. The step program for PCR was as follows: one cycle of 95° C., 5 min; 60° C., 3 min; 72° C., 3 min; followed by 35 cycles of 95° C., 15 s; 60° C., 15 s; 72° C., 30 s; followed by one cycle of 72° C., 5 min in a Robocycler (Stratagene, La Jolla, Calif.).

Ligation of parental PCR fragments: Each amplified product was ethanol-precipitated and dissolved in 10 μl dH₂O. Two microliters of each sample were fractionated on a 6% polyacrylamide gel for quantitation. Approximately 25 ng (1-6 μl) of each product were combined in a final volume of 20 μl and ligated for 16 h at 4° C. in 1×T4 ligase buffer with 3 Weiss U of T4 DNA ligase (NEB).

Chimeric amplification reaction: To produce the chimeric Flop-β-Flip PCR product, a PCR amplification was performed in 1×Taq buffer (10 mM Tris pH 9.0, 50 mM KCl, and 0.1% Triton X-100), supplemented with 2 mM MgCl₂, 200 μM of each dNTP, 0.01 U Pfu polymerase, and 5 U of Taq polymerase (Promega, Madison, Wis.). A 2 μl sample of the ligation mix (above) was used as a template, with 0.4 μM each of the 5′-Flop and 3′-Flip primers. The PCR program was identical to that for the parental reactions except that the annealing temperature was 61° C.

PCR amplification generating products with 3′-DNA overhangs: Parental PCR reactions. Three PCR reactions were performed to amplify Flop, Flip, and β-globin intron 1. Each 100 μl reaction contained 2.5 U of Pfu Turbo polymerase (Stratagene), 1× cloned Pfu buffer (10 mM (NH₄)₂SO₄, 20 mM Tris pH 8.8, 2 mM MgSO₄, 10 mM KCl, 0.1% Triton X-100, and 0.1 mg ml-1 bovine serum albumin), 200 μM of each dNTP, 1 mM MgSO₄, and primers (including alternative primers from Primer set 2) at a final concentration of 0.5 μM each. The Flop and Flip reactions contained 375 ng of human genomic DNA, while the β-globin reaction contained 5 ng of HβT7 DNA. The PCR step program was one cycle of 95° C., 5 min; 50° C., 3 min; 72° C., 3 min; followed by 40 cycles of 95° C., 30 s; 50° C., 30 s; 72° C., 45 s; followed by one cycle of 72° C., 5 min for the Flip and Flop fragments. The same program was used to amplify β-globin intron 1, except the annealing temperature was 46° C. The PCR was followed by an incubation at 72° C. for 30 min with 5 U of Tth polymerase (Epicentre Technologies, Madison, Wis.), to fill in the 5′-RNA overhangs. Note, in more recent experiments, M-MLV RT was used, rather than Tth, to fill in the overhangs. When M-MLV RT was used, the fragments were separated on agarose gels before treatment with 200 U of M-MLV RT (Life Technologies, Rockville, Md.) in 1× first-strand buffer (50 mM Tris pH 8.3, 75 mM KCl, 3 mM MgCl₂), 10 mM dithiothreitol, and 0.5 mM dNTPs in 20 μL.

Hydrolysis, phosphorylation and ligation of parental PCR fragments: The amplified parental PCR products were excised from an agarose gel and purified. Five microliters of each purified sample were fractionated on an agarose gel for quantitation. NaOH (1 N) was added to 8 μl of each of the gel-isolated fragments to a final concentration of 0.2 N, and the samples were incubated at 45° C. for 30 min. The base was neutralized by addition of 2 μl of 1 N HCl, and the DNA fragments were phosphorylated in 1×T4 ligase buffer (US Biochemicals, USB; Cleveland, Ohio) in a total of 20 μl for 30 min at 37° C. using 10 U of polynucleotide kinase (PNK) (USB). Approximately 25 ng (3-6 μl) of each phosphorylated product were combined in a final volume of 20 μl and ligated for 16 h at 14° C. in 1×T4 ligase buffer with 5 Weiss U of T4 DNA ligase (USB).

Chimeric amplification reaction: To produce the chimeric Flop-β-Flip product, a secondary PCR amplification was performed, as described above for the parental DOC reactions, using 1 μl of ligation reaction as template, the 5′-Flop and 3′-Flip primers, and an annealing temperature of 58° C.

Oligonucleotide primers. Chimeric RNA-DNA primers were purchased from Oligos, Etc. (Wilsonville, Oreg.). Ribonucleotide bases are shown in lowercase.

Primer set 1:

| | |
|---|---|
| 5′-Flop | (5′-AAATGCGGTTAACCTCGCAG-3′). |
| 3′-Flop | (5′-accuTGGAATCACCTCCCCC-3′). |
| 5′-β | (5′-agguTGGTATCAAGGTTACA-3′). |
| 3′-β | (5′-cuAAGGGTGGGAAAATAGAC-3′). |
| 5′-Flip | (5′-agAACCCCAGTAAATCTTGC-3′). |
| 3′-Flip | (5′-CTTACTTCCAGAGTCCTTGG-3′). |

Primer set 2:

Alternative primers were used in some DOC experiments.

| | |
|---|---|
| 3′-β | (5′-uucuAAGGGTGGGAAAATAG-3′). |
| 5′-Flip | (5′-agaaCCCCAGTAAATCTTGC-3′). |

Amplification templates. PCR amplification was used to generate a chimeric gene composed of two exons of the human glutamate receptor 2 (GluR2) gene linked to intron 1 of the human β-globin gene; GenBank under accession numbers: V00499 (β-globin intron 1), X64830 (Flop), X64829 (Flip). In each experiment, the intron and each of the exons was individually amplified. β globin intron 1 was always amplified using HβT7, a derivative of HβΔ6. Different templates were used for Flip and Flop in different experiments. Human GluR-B #7 contains a genomic fragment of the GluR2 gene that begins in exon 13 and ends in exon 16.

What is claimed is:

1. A method of generating a DNA molecule, the method comprising:
(a) hybridizing a plurality of oligonucleotides to a complementary portion of a nucleotide template, wherein the oligonucleotides comprise at least one non-natural nucleotide;
(b) coupling the plurality of hybridized oligonucleotides to form a first nucleotide strand;
(c) synthesizing a first complement to the first nucleotide strand, thereby generating a DNA molecule.

2. The method of claim 1, wherein synthesizing comprises reacting a mixture that comprises:
(a) the first nucleotide strand;
(b) at least one oligonucleotide primer that hybridizes to the first nucleotide strand;
(c) a polymerase; and
(d) a nucleotide mixture that comprises dATP, dCTP, dGTP, and dTTP.

3. The method of claim 2, wherein the nucleotide mixture does not comprise a non-natural nucleotide triphosphate.

4. The method of claim 1, wherein synthesizing comprises:
(a) hybridizing a second plurality of oligonucleotides to the first nucleotide strand; and
(b) coupling the second plurality of oligonucleotides to generate the first complement to the first nucleotide strand.

5. The method of claim 4, wherein the second plurality of oligonucleotides comprises at least one non-natural nucleotide that base-pairs with a corresponding non-natural nucleotide present in the first nucleotide strand.

6. The method claim 1, wherein coupling comprises enzymatic coupling.

7. The method of claim 7, wherein enzymatic coupling comprises ligation.

8. The method of claim 1, further comprising amplifying the DNA molecule.

9. The method of claim 8, wherein the DNA molecule is amplified in a reaction mixture that does not comprise a non-natural nucleotide triphosphate.

10. The method of claim 1, further comprising transforming or transfecting the DNA molecule into a cell and subjecting the cell to conditions suitable for replicating the DNA.

11. The method of claim 1, further comprising transforming or transfecting the DNA molecule into a cell and subjecting the cell to conditions suitable to select for expression of at least one gene product encoded by the DNA.

12. The method of claim 1, further comprising sequencing the double-stranded DNA.

13. The method of claim 1, wherein the at least one non-natural base is selected from isoguanine and isocytosine.

14. The method of claim 1, wherein synthesizing is performed in the presence of at least one non-natural nucleotide.

15. The method of claim 1, wherein the DNA molecule is no less than about 1000 nucleotides in length.

16. The method of claim 1, wherein hybridizing comprises base-pairing between at least one non-natural nucleotide in a first oligonucleotide and at least one non-natural nucleotide in a second oligonucleotide and base-pairing between at least one non-natural nucleotide in the first oligonucleotide and at least one non-natural nucleotide in a third oligonucleotide, and wherein the coupling occurs between the second and third oligonucleotides.

17. The method of claim 16, wherein the first oligonucleotide or the second oligonucleotide includes at least one non-natural base within 5 nucleotides of the 5′ or 3′ terminus of the oligonucleotide.

18. The method of claim 16, wherein the third oligonucleotide includes at least one non-natural base within 5 nucleotides of the 5′ or 3′ terminus of the oligonucleotide.

19. The method of claim 16, wherein:
(a) at least one of the first oligonucleotide and the second oligonucleotide are located at a first position on a solid substrate;
(b) the third oligonucleotide is located at a second position on the solid substrate adjacent to the first position; and
(c) the first position and second position are proximal and at a distance of no more than about 10 microns.

20. The method of claim 19, wherein at least one of the first oligonucleotide, the second oligonucleotide, and the third oligonucleotide is reversibly immobilized on the substrate.

21. The method of claim 19, wherein at least one of the first oligonucleotide, the second oligonucleotide, and the third oligonucleotide is irreversibly immobilized on the substrate.

22. The method of claim 19, wherein at least one of the first oligonucleotide, the second oligonucleotide, and the third oligonucleotide is covalently conjugated to the substrate.

23. A method for synthesizing a DNA molecule, the method comprising:
(a) replicating a DNA template that includes a first base pair at a selected position, wherein at least one base of the first base pair is a non-natural base;
(b) converting the at least one non-natural nucleotide of the first base pair to a selected natural nucleotide to generate a second base pair.

24. The method of claim 23, wherein the converting is by replicating the DNA template using a polymerase.

25. The method of claim 23, wherein the first base pair is selected from the group consisting of A:iC, iC:T, iG:iC, and iG:T, and the second base pair is A:T.

26. The method of claim 23, wherein the first base pair is selected from the group consisting of iC:iG, iC:iC, iG:iG, and A:iG and the second base pair is T:A.

27. The method of claim 23, wherein the first base pair is selected from the group consisting of iC:C, G:iC, and G:iG and the second base pair is G:C or T:A.

28. The method of claim 23, wherein the first base pair is iG:C and the second base pair is G:C.

29. The method of claim 23, wherein the replicating and converting occurs in a cell.

30. A method for synthesizing a DNA molecule comprising replicating a DNA template that includes at least one non-natural base in a cell.
